(19) United States
(12) Patent Application Publication
(10) Pub. No.: US 2023/0416801 A1
(43) Pub. Date: Dec. 28, 2023

(54) ANTIMICROBIC SUSCEPTIBILITY TESTING USING RECURRENT NEURAL NETWORKS

(71) Applicant: Beckman Coulter, Inc., Brea, CA (US)

(72) Inventor: Frederick Rupisan CUENCO, Antelope, CA (US)

(21) Appl. No.: 18/037,428

(22) PCT Filed: Nov. 18, 2021
(86) PCT No.: PCT/US21/59829

§ 371 Date: May 17, 2023

Related U.S. Application Data
(60) Provisional application No. 63/115,768, filed on Nov. 19, 2020.

(51) Int. Cl. C12Q 1/18; G16B 40/00

(52) U.S. Cl. CPC C12Q 1/18; G16B 40/00

(57) ABSTRACT

An optimized testing method is used to determine minimum inhibitory concentration (MIC) of a particular antimicrobic for use on a sample. This may include iteratively imaging wells inoculated with the sample and containing various concentrations of the antimicrobic. The images are thereafter processed to identify MIC based on sequences in information provided as input to a machine learning model.

### RELATED APPLICATION

This application is related to, and claims the benefit of, provisional patent application 63/115,768 titled “Antimicrobic Susceptibility Testing Using Recurrent Neural Networks,” filed in the United States Patent Office on Nov. 19, 2020. That application is hereby incorporated by reference in its entirety.

### BACKGROUND

Various types of tests related to patient diagnosis and therapy can be performed by analysis of the patient's microorganisms, or “microbes.” Microbes are microscopic living organisms such as bacteria, fungi, or viruses, which may be single-celled or multicellular. Biological samples containing the patient's microorganisms may be taken from a patient's infections, bodily fluids or abscesses and may be placed in test panels or arrays, combined with various reagents, incubated, and analyzed to aid in treatment of the patient. Automated biochemical analyzers have been developed to meet the needs of health care facilities and other institutions to facilitate analysis of patient samples and to improve the accuracy and reliability of assay results when compared to analysis using manual operations and aid in determining effectiveness of various antimicrobials. An antimicrobial is an agent that kills microorganisms or inhibits their growth, such as antibiotics which are used against bacteria and antifungals which are used against fungi. However, with ever changing bacterial genera and newly discovered antimicrobials, the demand for biochemical testing has increased in both complexity and volume.

An important family of automated microbiological analyzers function as a diagnostic tool for determining both the identity of an infecting microorganism and of an antimicrobic effective in controlling growth of the microorganism. Automated microbiological analyzers function as a diagnostic tool for determining both the identity of an infecting microorganism and of an antimicrobic effective in controlling growth of the microorganism. In performing the diagnostic tests, identification and in vitro antimicrobic susceptibility patterns of microorganisms isolated from biological samples are ascertained. Conventional versions of such analyzers may place a small sample to be tested into a plurality of small sample test wells in panels or arrays that contain different enzyme substrates or antimicrobics in serial dilutions. Identification (ID) testing of microorganisms, and antimicrobic susceptibility testing (AST) for determining Minimum Inhibitory Concentrations (MIC) of an antimicrobic effective against the microorganism may utilize color changes, fluorescence changes, the degree of cloudiness (turbidity) in the sample test wells created in the arrays, or other information derived from the testing. Both AST and ID measurements and subsequent analysis may be performed by computer controlled microbiological analyzers to provide advantages in reproducibility, reduction in processing time, avoidance of transcription errors and standardization for all tests run in the laboratory.

In ID testing of a microorganism, a standardized dilution of the patient's microorganism sample, known as an inoculum, is first prepared in order to provide a bacterial or cellular suspension having a predetermined known concentration. This inoculum is placed in a plurality of test wells that may contain or thereafter be supplied with predetermined test media. Depending on the species of microorganism present, this media will facilitate changes in color, turbidity, fluorescence, or other characteristics after incubation. These changes are used to identify the microorganism in ID testing.

In AST testing, a plurality of test wells are filled with inoculum and increasing concentrations of a number of different antimicrobial agents, for example antibiotics. The different antimicrobial agents may be diluted in a growth medium or liquid medium to concentrations that include those of clinical interest. After incubation, the turbidity will be increased or unchanged in test wells where growth has not been inhibited by the antimicrobics in those test wells. The MIC of each antimicrobial agent is measured by lack of growth with respect to each concentration of antimicrobial agent. It follows that the lowest concentration of antimicrobial agent displaying a lack of growth is the MIC.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1A depicts a portion of a diagrammatic view of an exemplary biological testing system;

FIG. 1B depicts another portion of the diagrammatic view of the biological testing system of FIG. 1A;

FIG. 2 depicts a perspective view of an exemplary incubator system and an exemplary optics system of the biological testing system of FIG. 1B;

FIG. 3 depicts a perspective view of the optics system of FIG. 2;

FIG. 4 depicts another perspective view of the optics system of FIG. 2 showing an XY stage of the optics system;

FIG. 5 depicts a diagrammatic view of portions of the optics system of FIG. 2;

FIG. 6 depicts a diagrammatic view of an exemplary computer system;

FIG. 7 depicts a chart of theoretical growth curves for a microbe;

FIG. 8 depicts a schematic view of an exemplary growth test well for use in the biological testing system of FIGS. 1A and 1B;

FIG. 9 depicts an exemplary image analysis cycle for use in the biological testing system of FIGS. 1A and 1B;

FIG. 10 depicts an exemplary raw image captured by the optics system of FIG. 2;

FIG. 11 depicts an exemplary enhanced image derived from the raw image of FIG. 10;

FIG. 12 depicts an exemplary gradient image derived from the raw image of FIG. 10;

FIG. 13 depicts an exemplary image enhancement flowchart;

FIG. 14 depicts an exemplary dynamic image enhancement flowchart;

FIG. 15 depicts an exemplary segmented image derived from the raw image of FIG. 10;

FIG. 16 depicts an exemplary image segmentation flowchart;

FIG. 17 depicts an exemplary optimized antimicrobic susceptibility testing method of the present invention;

FIG. 18 depicts an exemplary architecture for a machine learning model that some embodiments may use to apply recurrent neural networks to the task of MIC identification;

FIG. 19 depicts an individual GRU that could be used to recognize a particular growth pattern;

FIG. 20 depicts a flowchart showing processing a GRU could perform; and

FIG. 21 depicts an architecture that could be used to train a model to generate microorganism counts.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### SUMMARY

Various aspects and embodiments of the invention are set out in the claims appended hereto.

In a first aspect of the invention, there is provided a method comprising: (a) creating a plurality of test mixtures in a plurality of test wells, wherein: (i) each test mixture from the plurality of test mixtures is inoculated using a biological sample; (ii) each test mixture from the plurality of test mixtures comprises an antimicrobial solution comprising an antimicrobial agent; (iii) in each test mixture from the plurality of test mixtures, the antimicrobial solution in that test mixture differs from the antimicrobial solution in each other test mixture from the plurality of test mixtures; and (iv) the same biological sample is used to inoculate each test mixture from the plurality of test mixtures; (b) incubating each of the test mixtures; (c) for each test mixture from the plurality of test mixtures, at a plurality of imaging times, wherein each of the imaging times takes place after incubation has begun, capturing an image of that test mixture; (d) obtaining a plurality of growth predictions by performing steps comprising, for each test mixture from the plurality of test mixtures, providing a data sequence to a machine learning model, wherein: (i) the data sequence comprises a plurality of input items; (ii) each input item from the plurality of input items corresponds to an imaging time from the plurality of imaging times; and (iii) the machine learning model is adapted to recognize, and to make growth predictions based on, temporal sequences; and (e) generating a minimum inhibitory concentration, MIC, determination for the biological sample based on the plurality of growth predictions.

In another aspect of the invention, there is provided a biological testing system comprising a processor configured with a set of computer instructions operable, when executed, to cause the system to perform a method comprising: (a) creating a plurality of test mixtures in a plurality of test wells, wherein: (i) each test mixture from the plurality of test mixtures is inoculated using a biological sample; (ii) each test mixture from the plurality of test mixtures comprises an antimicrobial solution comprising an antimicrobial agent; (iii) in each test mixture from the plurality of test mixtures, the antimicrobial solution in that test mixture differs from the antimicrobial solution in each other test mixture from the plurality of test mixtures; and (iv) the same biological sample is used to inoculate each test mixture from the plurality of test mixtures; (b) incubating each of the test mixtures; (c) for each test mixture from the plurality of test mixtures, at a plurality of imaging times, wherein each of the imaging times takes place after incubation has begun, capturing an image of that test mixture; (d) obtaining a plurality of growth predictions by performing steps comprising, for each test mixture from the plurality of test mixtures, providing a data sequence to a machine learning model, wherein: (i) the data sequence comprises a plurality of input items; (ii) each input item from the plurality of input items corresponds to an imaging time from the plurality of imaging times; and (iii) the machine learning model is adapted to recognize, and to make growth predictions based on, temporal sequences; and (e) generating a minimum inhibitory concentration, MIC, determination for the biological sample based on the plurality of growth predictions.

In another aspect of the invention, there is provided a computer program product comprising a non-transitory computer readable medium having stored thereon a set of computer instructions operable, when executed, to cause a biological testing system to perform a method comprising: (a) creating a plurality of test mixtures in a plurality of test wells, wherein: (i) each test mixture from the plurality of test mixtures is inoculated using a biological sample; (ii) each test mixture from the plurality of test mixtures comprises an antimicrobial solution comprising an antimicrobial agent; (iii) in each test mixture from the plurality of test mixtures, the antimicrobial solution in that test mixture differs from the antimicrobial solution in each other test mixture from the plurality of test mixtures; and (iv) the same biological sample is used to inoculate each test mixture from the plurality of test mixtures; (b) incubating each of the test mixtures; (c) for each test mixture from the plurality of test mixtures, at a plurality of imaging times, wherein each of the imaging times takes place after incubation has begun, capturing an image of that test mixture; (d) obtaining a plurality of growth predictions by performing steps comprising, for each test mixture from the plurality of test mixtures, providing a data sequence to a machine learning model, wherein: (i) the data sequence comprises a plurality of input items; (ii) each input item from the plurality of input items corresponds to an imaging time from the plurality of imaging times; and (iii) the machine learning model is adapted to recognize, and to make growth predictions based on, temporal sequences; and (e) generating a minimum inhibitory concentration, MIC, determination for the biological sample based on the plurality of growth predictions.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. BIOLOGICAL TESTING SYSTEM HARDWARE

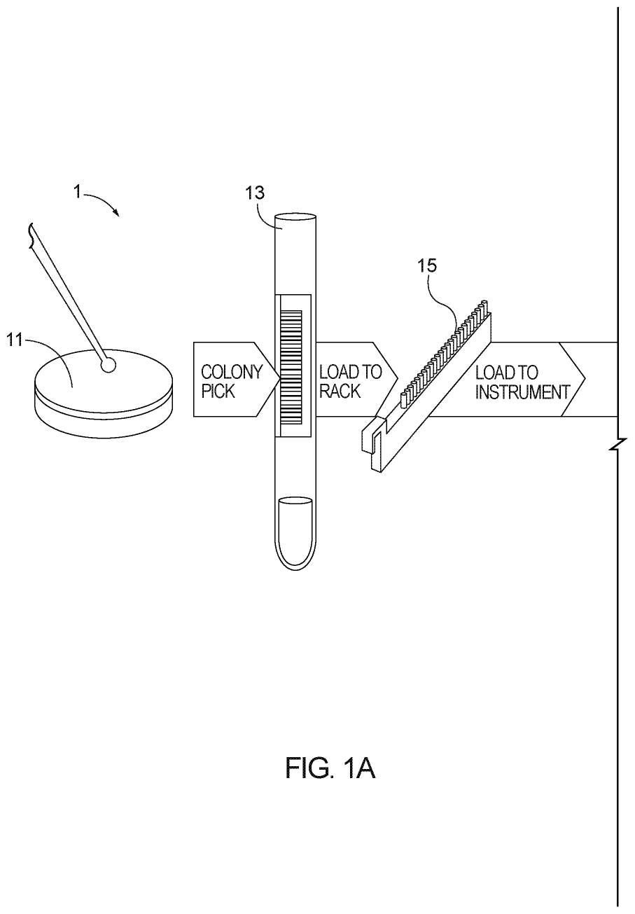

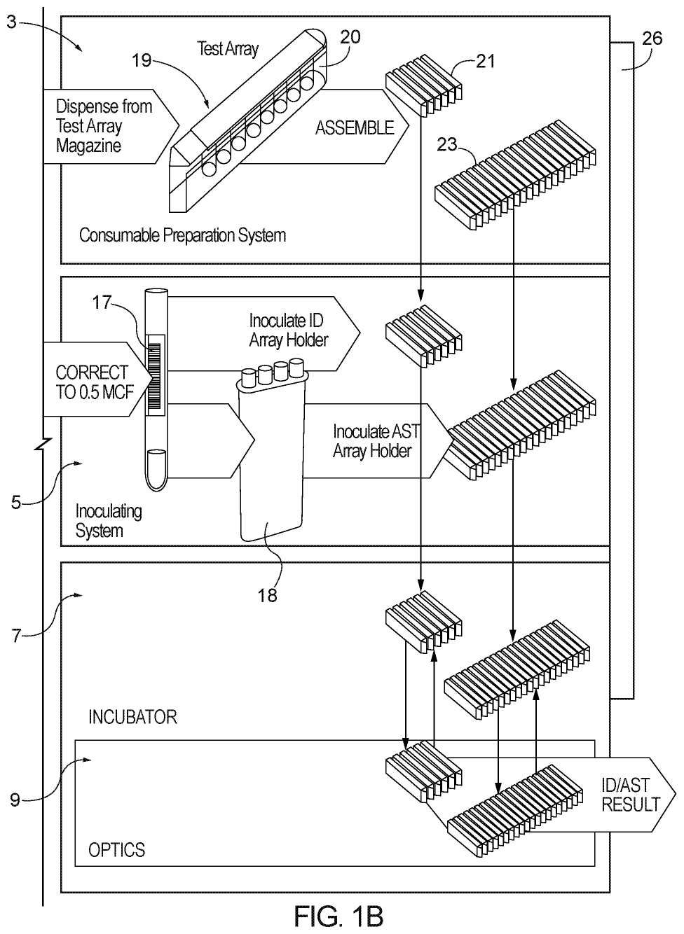

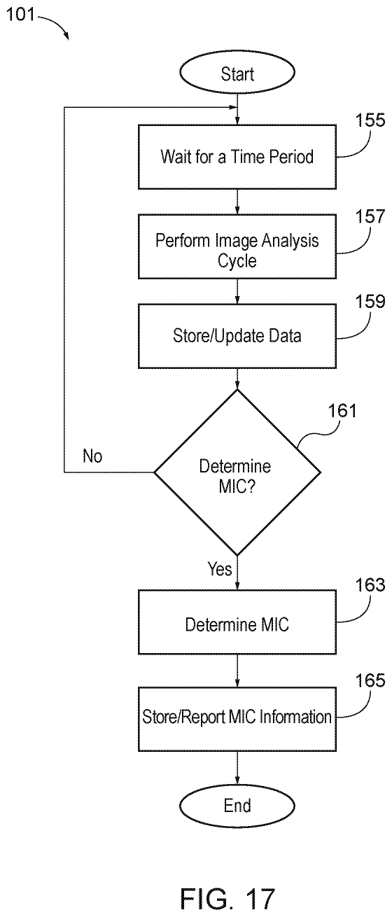

FIGS. 1A and 1B depict a diagrammatical example of various hardware components available in a biological testing system 1. Biological testing system 1 facilitates an optimized antimicrobial susceptibility testing (AST) method 101 (FIG. 17). Biological testing system 1 broadly includes a consumable preparation system 3, an inoculating system 5, an incubator system 7, and an optics system 9. The various systems within biological testing system 1 coordinate with each other and work automatically once loaded with adequate material by a user.

To operate biological testing system 1, the user first acquires an appropriate microbe sample. As shown in FIG. 1A, a microbe sample may be obtained from an agar plate 11 or, under certain circumstances, from a blood sample. Next, the user prepares an inoculum suspension by transferring the microbes into a tube containing a suitable liquid medium or broth. One such tube is shown in FIG. 1A as an inoculum 13. In some versions of biological testing system 1, the liquid medium or broth may be an approximately 0.5 mM phosphate buffered solution with small amounts of sodium and potassium chloride to aid in maintaining the viability of the microbes introduced into solution without adversely interfering with the MIC determination or other associated testing. The phosphate buffered solution may be used in both ID testing and AST testing to minimize the need for different inoculum across the two systems and leverage the efficiencies in having a single broth. Each inoculum 13 is placed into an inoculum rack 15 and the entire inoculum rack 15 is placed into inoculating system 5. Once in inoculating system 5, the inoculum in each inoculum 13 is adjusted if necessary to a standard turbidity value of 0.5 McFarland to create an inoculum 17. In some versions of biological testing system 1, a 1 microliter plastic loop or swab may be provided to the user to easily pick colonies from the agar plate and to minimize the amount of adjustment needed to bring the inoculum to the desired turbidity value. Once adjusted to the desired turbidity value, the inoculum is finalized. The finalized inoculum will be referred to hereinafter as inoculum 17, as depicted in FIG. 1B. Inoculum 17 may be further diluted into a 1:250 dilution and converted into an inoculum 18. As will be discussed in more detail below, the inoculum contained in each inoculum 17 is applied to an identification (ID) array holder 21, while the inoculum contained in each inoculum 18 is applied to an AST array holder 23. Both ID array holder 21 and AST array holder 23 are assembled by consumable preparation system 3 and provided to inoculating system 5 for use with inoculum 17 and inoculum 18.

Consumable preparation system 3 is loaded with magazines of test arrays 19, which may contain various antimicrobials or other agents required by biological testing system 1 disposed in a series of test wells 20. For example, test array 19 may comprise an antimicrobic dilution array or an identification array. Consumable preparation system 3 may also be loaded with bulk diluents (not shown) and/or various other elements for preparing and finalizing ID array holder 21 and AST array holder 23 and the inoculate therein. Primarily, consumable preparation system 3 operates to retrieve test arrays 19 as required and combine each retrieved test array 19 into either ID array holder 21 or AST array holder 23. Test arrays 19 may be selected and assembled by a robotic gripper (not shown) or other mechanical features as dictated by the prescribed testing. For example, a physician may order biological testing using the antibiotic amoxicillin. Test arrays 19 relating to amoxicillin testing are therefore retrieved and assembled into the appropriate ID array holder 21 and AST array holder 23. All or some portions of test array 19 may be formed of a styrene material to aid in reducing fluorescent crosstalk, fallout, and/or bubbles when digitally examining each test well 20.

Once inoculum 17, inoculum 18, ID array holder 21, and AST array holder 23 are assembled, inoculating system 5 dispenses the generally undiluted inoculum from inoculum 17 into test wells 20 of ID array holder 21 and the diluted inoculum from inoculum 18 into test wells 20 of AST array holder 23. The time between applying inoculum 17 to ID array holder 21 or inoculum 18 to AST array holder 23 and the start of logarithmic growth of the microbes disposed therein is known as “lag time.” Lag time may be decreased by using enhanced broth such as a broth with yeast extract, vitamins, and/or minerals. Lag time may also be decreased by increasing the inoculum. In some versions of biological testing system 1, the amount of inoculum may be doubled to decrease the lag time by approximately 30 minutes without affecting the accuracy of the MIC determination. The dispensing may be accomplished via an elevator assembly 26 having an XY robot or XYZ robot (not shown) with a gripper (not shown) and pipettor (not shown), along with various circuitry, channels, and tubing as necessary. The XYZ robot is tasked with retrieving inoculum from inoculum racks 15 and dispensing the inoculum into test wells 20 of ID array holder 21 and AST array holder 23. Once ID array holder 21 and AST array holder 23 are sufficiently loaded with inoculum, each ID array holder 21 and AST array holder 23 are moved into incubator system 7 by way of an elevator assembly 26.

The XY robot or XYZ robot may be, or include, a Cartesian coordinate robot or gantry robot which comprises three principal axes (x-axis, y-axis and z-axis) of control which are linear. That is, the robot generally moves in a straight line rather than rotates along axes which are perpendicular (at right angles to) each other. A Cartesian coordinate robot or gantry robot may comprise three sliding joints that correspond to movement up-down (the z-axis), in-out (the y-axis), and back-forth (the z-axis), with respect to a plane or object. Alternatively, the elevator assembly may comprise any of an Articulate robot, a Cylindrical coordinate robot, a Spherical coordinate robot, a SCARA (Selective Compliance Assembly Robot Arm) robot, and/or serial manipulators.

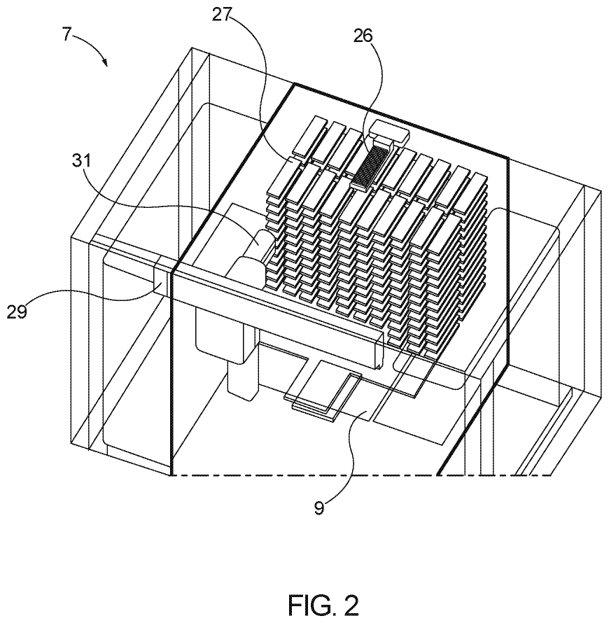

As shown in FIG. 2, incubator system 7 includes slots 27 for holding a large number of ID array holders 21 and AST array holders 23. Each array holder is placed into a corresponding slot 27 by an XYZ robot 29 using a gripper 31. XYZ robot 29 operates to move in any portion of the XYZ plane and position gripper 31 proximate the desired ID array holder 21 or AST array holder 23. While in incubator system 7, each array holder incubates in specific desired environmental conditions. For example, incubator system 7 may be set to incubate array holders at thirty-five degrees Celsius. At certain time intervals during the incubation, XYZ robot 29 retrieves a particular ID array holder 21 or AST array holder 23 and move the selected array holder into the optics system 9.

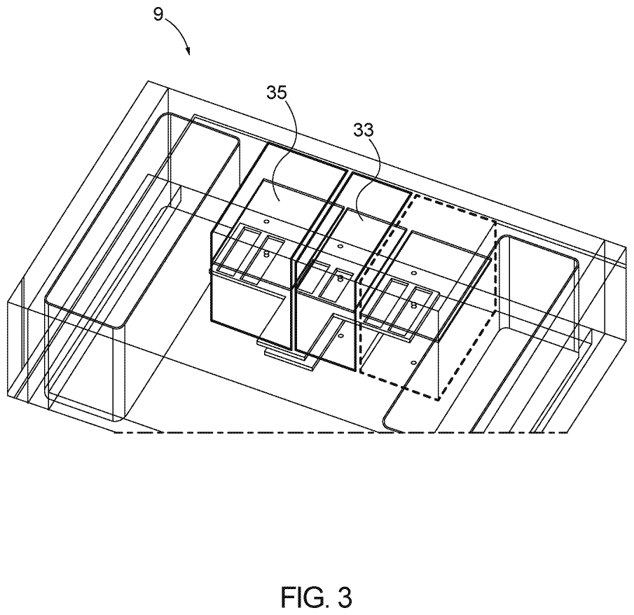

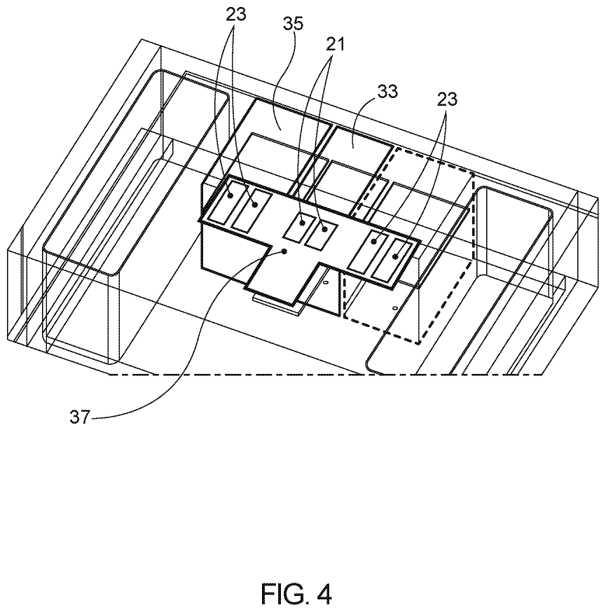

As shown in FIG. 2-4, optics system 9 includes features that are configured to observe, monitor, review, and/or capture images for each test well 20 of an ID array holder 21 or AST array holder 23. Specifically, each ID array holder 21 is monitored by an ID fluorimeter 33, and each AST array holder 23 is monitored by an AST camera 35. To accomplish the monitoring, XYZ robot 29 retrieves the particular array holder with gripper 31 and places the selected array holder onto an XY-stage 37. The XY-stage 37 moves in the XY plane to position the array holder under the associated monitoring element, namely, the ID array holders 21 are disposed under the ID fluorimeter 33 and the AST array holders 23 are disposed under the AST camera 35 for monitoring and observation in optics system 9. XY-stage 37 includes finely tuned motor control to allow each test well 20 of the associated array holder to be positioned accurately within the observation frame of either ID fluorimeter 33 or AST camera 35.

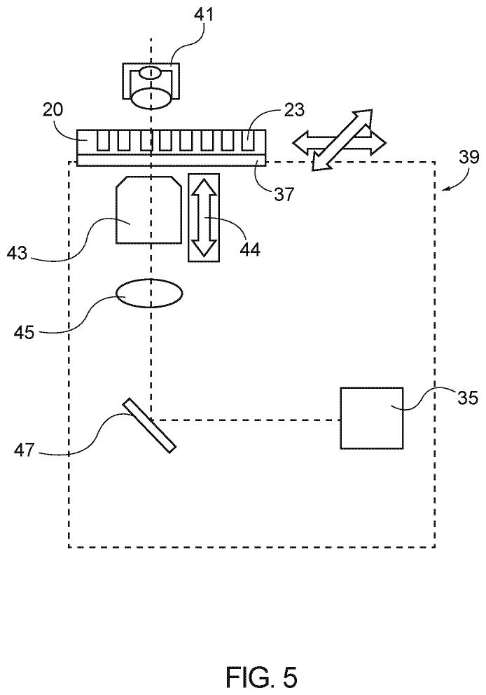

FIG. 5 illustrates an exemplary architecture for an AST optics portion 39 of optics system 9. AST optics portion 39 includes an illumination source 41, an objective lens 43, a tube lens 45, and a fold mirror 47. Illumination source 41 may comprise a condenser LED system for providing monochromatic illumination of each test well 20 of AST array holder 23. Objective lens 43 may comprise a Nikon 20×0.45 NA ELWD objective lens, an Olympus 10× objective lens or any other suitable kind of lens. Objective lens 43 may comprise a 20× objective lens with each pixel covering about 0.33 microns. A 20× objective lens provides both the ability to detect a reasonable number of cells at the beginning of cell growth (around 100-200 cells) and the ability to detect cell morphology. Objective lens 43 may comprise a 10× objective lens and/or a 5 MP camera for a larger dynamic range and/or a bigger sample of each test well 20) while maintaining enough resolution to count the microbes therein. The magnification of Objective lens 43 may be in the range of 5× to 50×. The selection of the magnification of the Objective lens 43 may depend on various factors, such as the size of an image sensor or the camera being used, the size of the microbes being photographed, the required pixel size or resolution of the captured image, and the like. In some exemplary embodiments of optics system 9, only one picture or image per test well 20 per pass is acquired by objective lens 43. Objective lens 43 may focus slightly off the bottom of test well 20 to eliminate background noise from the bottom of test well 20. In some versions of optics system 9, objective lens 43 is configured to focus approximately 5-10 microns from the bottom of test well 20. In some versions of optics system 9, objective lens 43 is configured to focus 8 microns from the bottom of test well 20. Objective lens 43 may also include a Z-stage 44 for allowing objective lens 43 to move in the Z-axis, relative to XY-stage 37. Thus, between XY-stage 37 moving AST array holder 23 in the XY plane and Z-stage 44 moving objective lens 43 in the Z-axis, each test well 20 of array holder 23 may be moved in any three-dimensional space to precisely align test wells 20 with the frame of AST camera 35. Tube lens 45 may be embodied in an achromatic tube lens. AST camera 35 may comprise a Sony IMX253 camera, various types of 5 megapixel cameras provided by other manufacturers such as Canon, Thorlabs, or Sentech, or any other suitable kind of camera. In some versions of optics system 9, XY-stage 37 and Z-stage 44 are replaced with an XYZ-stage to provide all three axes of three-dimensional movement of test wells 20.

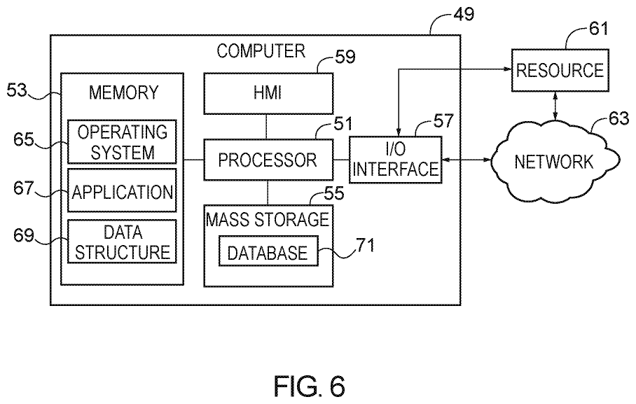

Referring now to FIG. 6, the various components of biological testing system 1 may incorporate one or more computing devices or systems, such as exemplary computer system 49. For example, any one of consumable preparation system 3, inoculating system 5, incubator system 7, and/or optics system 9 may incorporate one or more computing systems such as exemplary computer system 49. Alternatively, each of these subsystems of biological testing system 1 may function via commands from one overall computing system such as exemplary computer system 49.

Computer system 49 may include a processor 51, a memory 53, a mass storage memory device 55, an input/output (I/O) interface 57, and a Human Machine Interface (HMI) 59. Computer system 49 may also be operatively coupled to one or more external resources 61 via a network 63 or I/O interface 57. External resources may include, but are not limited to, servers, databases, mass storage devices, peripheral devices, cloud-based network services, or any other suitable computer resource that may used by computer system 49.

Processor 51 may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on operational instructions that are stored in memory 53. Memory 53 may include a single memory device or a plurality of memory devices including, but not limited, to read-only memory (ROM), random access memory (RAM), volatile memory, non-volatile memory, static random access memory (SRAM), dynamic random access memory (DRAM), flash memory, cache memory, or any other device capable of storing information. Mass storage memory device 55 may include data storage devices such as a hard drive, optical drive, tape drive, non-volatile solid state device, or any other device capable of storing information.

Processor 51 may operate under the control of an operating system 65 that resides in memory 53. Operating system 65 may manage computer resources so that computer program code embodied as one or more computer software applications, such as an application 67 residing in memory 53, may have instructions executed by the processor 51. In an alternative embodiment, processor 51 may execute application 67 directly, in which case the operating system 65 may be omitted. One or more data structures 69 may also reside in memory 53, and may be used by processor 51, operating system 65, or application 67 to store or manipulate data.

The I/O interface 57 may provide a machine interface that operatively couples processor 51 to other devices and systems, such as network 63 or external resource 61. Application 67 may thereby work cooperatively with network 63 or external resource 61 by communicating via I/O interface 57 to provide the various features, functions, applications, processes, or modules comprising embodiments of the invention. Application 67 may also have program code that is executed by one or more external resources 61, or otherwise rely on functions or signals provided by other system or network components external to computer system 49. Indeed, given the nearly endless hardware and software configurations possible, persons having ordinary skill in the art will understand that different versions of the invention may include applications that are located externally to computer system 49, distributed among multiple computers or other external resources 61, or provided by computing resources (hardware and software) that are provided as a service over network 63, such as a cloud computing service.

HMI 59 may be operatively coupled to processor 51 of computer system 49 in a known manner to allow a user to interact directly with the computer system 49. HMI 59 may include video or alphanumeric displays, a touch screen, a speaker, and any other suitable audio and visual indicators capable of providing data to the user. HMI 59 may also include input devices and controls such as an alphanumeric keyboard, a pointing device, keypads, pushbuttons, control knobs, microphones, etc., capable of accepting commands or input from the user and transmitting the entered input to the processor 51.

A database 71 may reside on mass storage memory device 55, and may be used to collect and organize data used by the various systems and modules described herein. Database 71 may include data and supporting data structures that store and organize the data. In particular, database 71 may be arranged with any database organization or structure including, but not limited to, a relational database, a hierarchical database, a network database, or combinations thereof. A database management system in the form of a computer software application executing as instructions on processor 51 may be used to access the information or data stored in records of the database 71 in response to a query, where a query may be dynamically determined and executed by operating system 65, other applications 67, or one or more modules.

### II. OPTIMIZED AST SYSTEM AND METHOD

In some versions, system 1 as discussed above may be used to facilitate some or all of the features provided in optimized AST method 101 such as shown in FIG. 17.

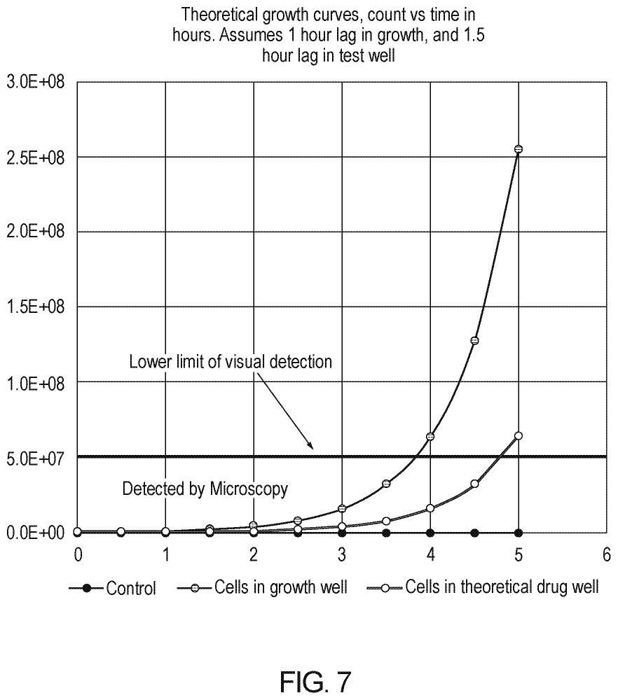

In some conventional processes, MIC is determined through manual visual inspection of test wells after waiting a period to allow the microbes to grow. However, as shown in FIG. 7, traditional methods of determining MIC are limited by what a human can visually perceive relative to the growth of the microbes within the test wells. Further, the presence of antimicrobic dilution concentrations that are below the MIC concentration may slow the growth rate, and take even longer to perceive. As shown in FIG. 7, the first six to seven doublings of the microbial sample cannot be observed visually by the human eye. However, the information provided through these initial doublings is often indicative of the MIC. As shown in FIGS. 7-20, optimized AST method 101 utilizes digital microscopy to monitor the count of microbes in one more test wells from the point of inoculation and thus allows for more rapid and accurate detection of the MIC.

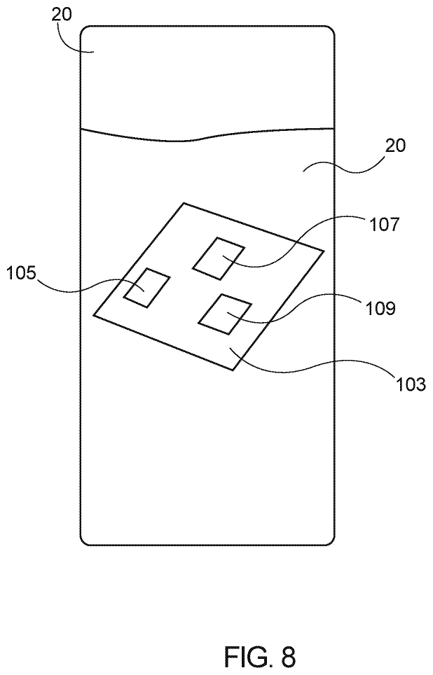

FIG. 8 provides an illustration of how data that can be used in an optimized AST method 101 can be captured from a test well. Test well 20 is embodied by a “384” style test well, having a clear viewing bottom. The volume of inoculum in test well 20 may be set to 20 microliters to reduce the amount of materials such as bulk diluents required by AST method 101 and/or minimize light artifacts and provide sampling of a consistent number of microbes from the 1:250 dilution of the 0.5 MacFarland inoculum. Decreasing the volume of inoculum in test well 20 generally increases the light artifacts. In some instances, capturing a single vertical plane 103 at 20× objective whereby AST camera 35 is set at 0.33 microns/pixel may be sufficient for sampling the inoculum and ensuring that each individual microbe is recognizable. However, these parameters are configurable and may change as desired by the user or the underlying needs of the system. Capturing three focal sites spaced about 5 microns apart within single vertical plane 103 may also provide a sufficient number of microbes in the sample to count for use in optimized AST method 101. These three focal sites are labeled site 105, site 107, and site 109 in FIG. 8. In an exemplary version of optimized AST method 101, each focal site is approximately 700×700 microns within single vertical plane 103, rather than capturing three sites in three different vertical planes. AST camera 35, optics system 9, and computer 49 are configured to capture an image of each focal site in consecutive time periods, manipulate each of these images, and thereafter use the data derived from these manipulated images to make a MIC determination.

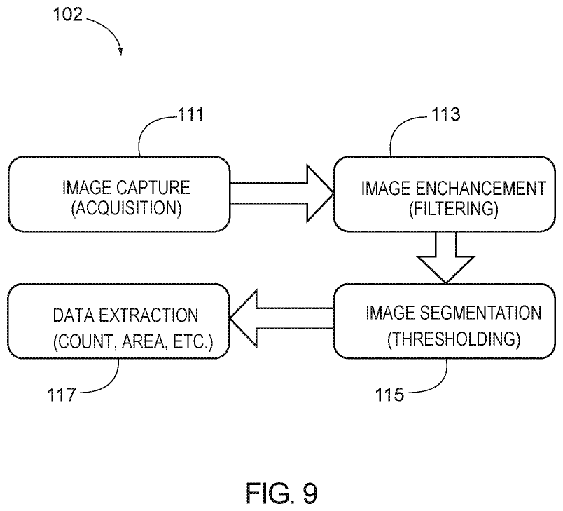

Image analysis cycle 102 is generally depicted in FIG. 9 and comprises an image capture step 111, an image enhancement step 113, an image segmentation step 115, and an object counting step 117. Optimized AST method 101 may include performing image analysis cycle 102 repetitively until a MIC is determined. Image analysis cycle 102 utilizes one or more instances of computer 49 and the various elements thereof to perform image capture step 111, image enhancement step 113, image segmentation step 115, and data extraction step 117, as well as any sub steps provided therein.

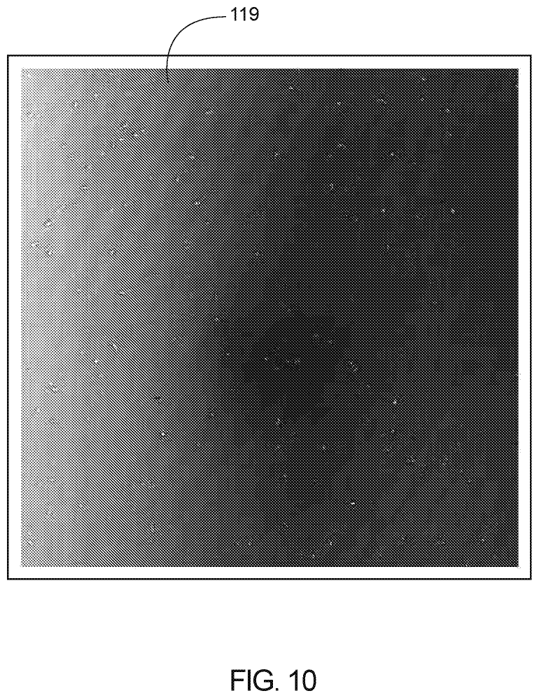

Image analysis cycle 102 begins with image capture step 111 captures a raw image 119 (FIG. 10) of the inoculum via AST camera 35 of optics system 9 and stores raw image 119 in memory 53. Raw image 119 may be considered to be an image which has not had any static or dynamic enhancement, filters, noise reduction or any other image correction. While raw image 119 is depicted as a single image, raw image 119 may be a composite of several separate images taken in vertical plane 103, for example, a composite of site 105, site 107, and site 109. Raw image 119 may also be a composite of several images taken in different planes within the inoculum sample or may be a single image. As illustrated in FIG. 10, raw image 119 may include deficiencies such as uneven illumination. Uneven illumination may be a result of the meniscus created by the inoculum in combination with the walls of the associated test well 20, which may affect the path of illumination source 41. Uneven background intensity may also be caused by environmental issues such as plastic deformation or from a variety of other sources. After raw image 119 is captured in image capture step 111, image capture step 111 moves to image enhancement step 113.

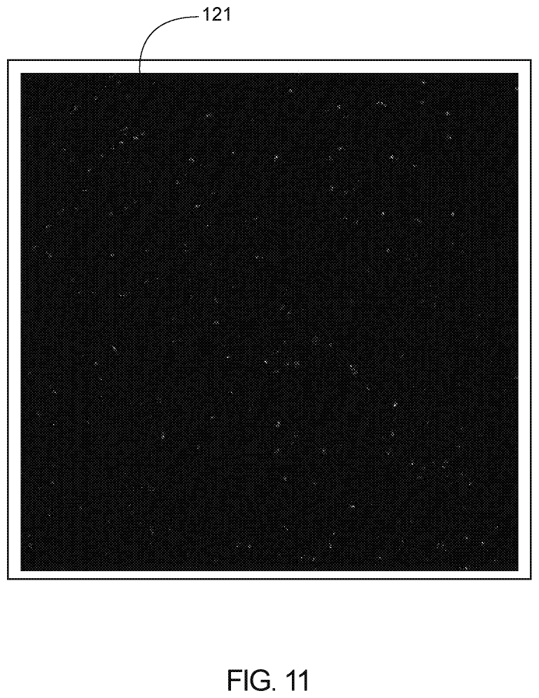

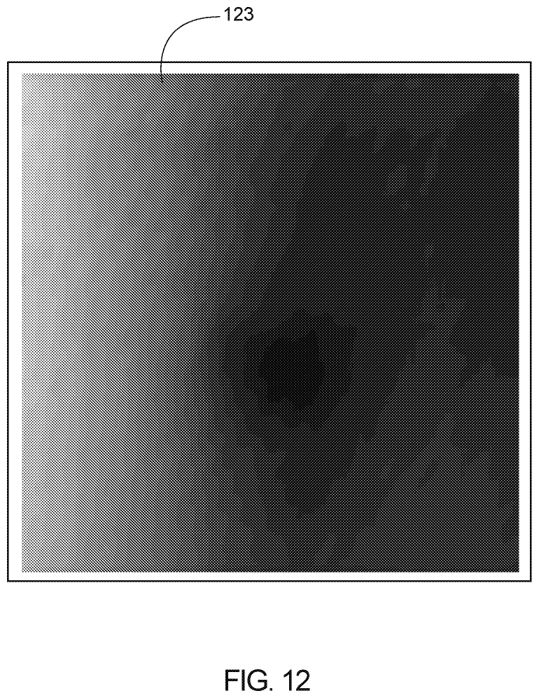

As shown in FIGS. 9, and 11-14, image enhancement step 113 processes raw image 119 to create an enhanced image 121 (FIG. 11), whereby enhanced image 121 is more suitable for the process of counting and recognizing microbes such as bacteria. During image enhancement step 113, one or more attributes of raw image 119 are modified. These attributes may include basic gray level transformations, noise filtering, and median filtering. For example, to resolve the problem of non-uniform illumination, a median filter may be applied to raw image 119 to arrive at a gradient image 123 (FIG. 12). This may be accomplished by selecting a pixel radius sufficient to provide a resulting image containing only the gradient of the background illumination of raw image 119. Gradient image 123 is then subtracted from raw image 119 to correct for the uneven illumination and generate enhanced image 121 free of uneven illumination.

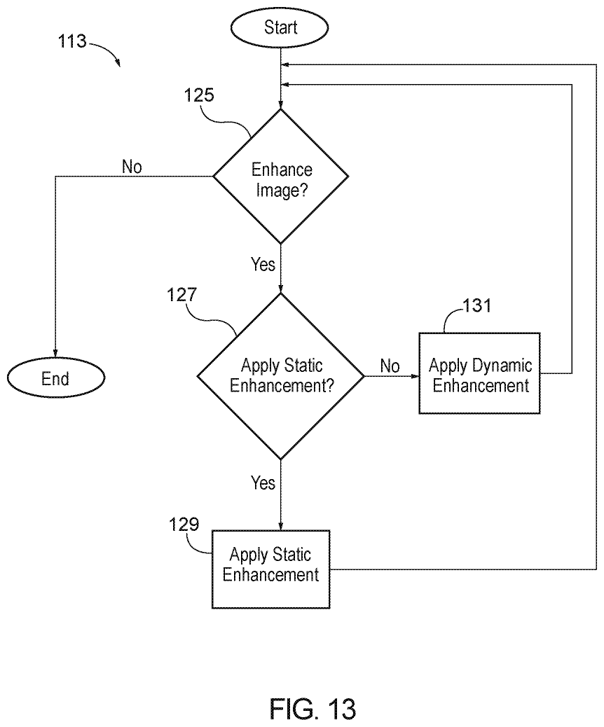

Image enhancement step 113 may apply image enhancements either statically, dynamically, or both. For example, the pixel radius for the median filter may be a statically set constant value or may be adaptively derived dynamically from characteristics of each raw image 119 captured through optics system 9. As shown in FIG. 13, some versions of image enhancement step 113 may include a step 125, whereby a determination is made as to whether the working image should undergo enhancement. If step 125 determines the working image should be enhanced, step 125 proceeds to a step 127. In step 127, a determination is made as to whether a static enhancement should be applied. If step 127 determines a static enhancement should be applied, step 127 proceeds to a step 129. If step 127 determines a static enhancement should not be applied, step 127 proceeds to a step 131. In step 127, a static enhancement is applied to the working image and step 127 proceeds back to step 125. In step 131, a dynamic enhancement is applied to the working image and step 131 proceeds back to step 125. If step 125 determines the working image should not be further enhanced, step 125 proceeds to end.

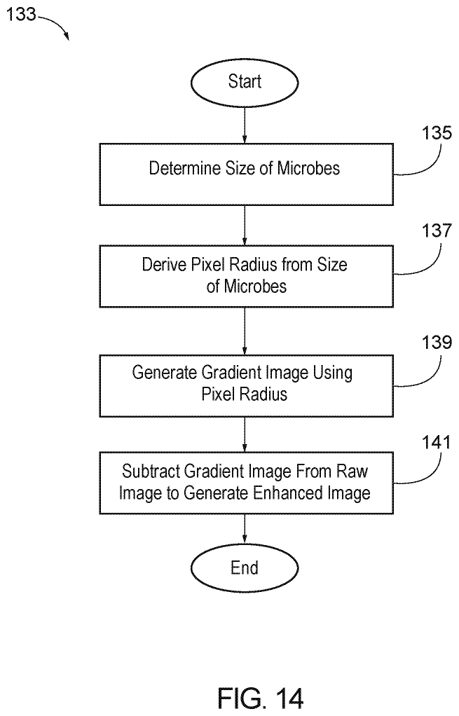

FIG. 14 illustrates an example of a method of dynamic image enhancement 133. Method of dynamic image enhancement 133 is directed to dynamically determining the appropriate pixel radius for use in a median filter enhancement. Method of dynamic image enhancement 133 begins with a step 135, whereby the size of the microbes depicted in raw image 119 is determined. The size of the microbes in raw image 119 may change depending on various circumstances and parameters associated with the inoculum and the overall optics system 9. Thus, while the literal size of the particular microbe being tested is generally constant in nature, the relative size of the microbes depicted in raw image 119 is dynamic and variable because of differences in parameters such as the lens objectification. Once step 135 determines the size of the microbes in raw image 119, step 135 proceeds to a step 137. In step 137, the pixel radius is derived from the determined size of the microbes. As a general example, if step 135 determines the maximum length of any given microbe in raw image 119 is five pixels, the pixel radius may be determined to be greater than five so that the filtered image only represents the gradient contained in the background. After step 135 derives the pixel radius based off the dynamically determined size of the microbes, step 135 proceeds to step 139. In step 139, gradient image 123 is generated based on processing raw image 119 with the derived pixel radius. After gradient image 123 is generated, step 139 moves to a step 141. In step 141, gradient image 123 is subtracted from raw image 119 to generate enhanced image 121. Thereafter, method of dynamic image enhancement 133 proceeds to end.

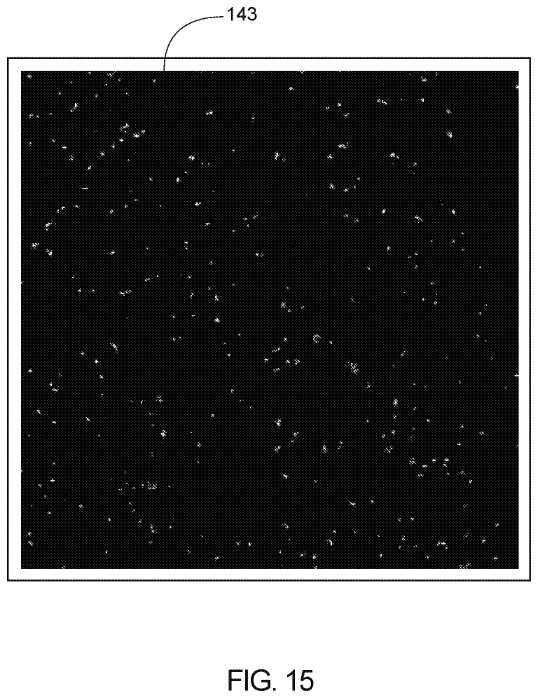

As shown in FIGS. 9, 15, and 16, image segmentation step 115 is used to partition the image into distinct regions containing pixels representing either the microbes as the foreground or the background. Image segmentation step 115 converts enhanced image 121 into a segmented image 143, as shown in FIG. 15. Image segmentation step 115 produces a binary image from enhanced image 121, where every pixel is equal to a value of either 0 or 1, where 0 refers to the background and 1 refers to a portion of a particular microbe.

Image artifacts such as noise may be removed by applying a noise reduction filter prior to applying the segmentation algorithm. Segmentation can be obtained using static threshold value or using an adaptive image thresholding method such as the Otsu cluster based thresholding algorithm. In this algorithm, the gray-level samples are clustered in two parts as background and foreground (object), or alternatively are modeled as a mixture of two Gaussians. The threshold value for the particular image thresholding algorithm used may be determined dynamically, depending on the overall image provided to image segmentation step 115 and the relative grayscale levels of the image. For example, inoculating system 5 or another element of system 1 may be configured to apply nigrosin to each test well 20 to enhance the image, as nigrosin does not attach to certain microbes such as bacteria. This may alter the relative greyscale levels in raw image 119 and require a different threshold value for the segmentation algorithm, as compared to a raw image 119 without nigrosin. In some versions of optimized AST method 101, threshold values may be determined dynamically by searching for edges within several areas of the image. These edges are the transition point between the background and a microbe. Thus, the threshold value can then be calculated as the average greyscale value for pixels on each side of the located edge.

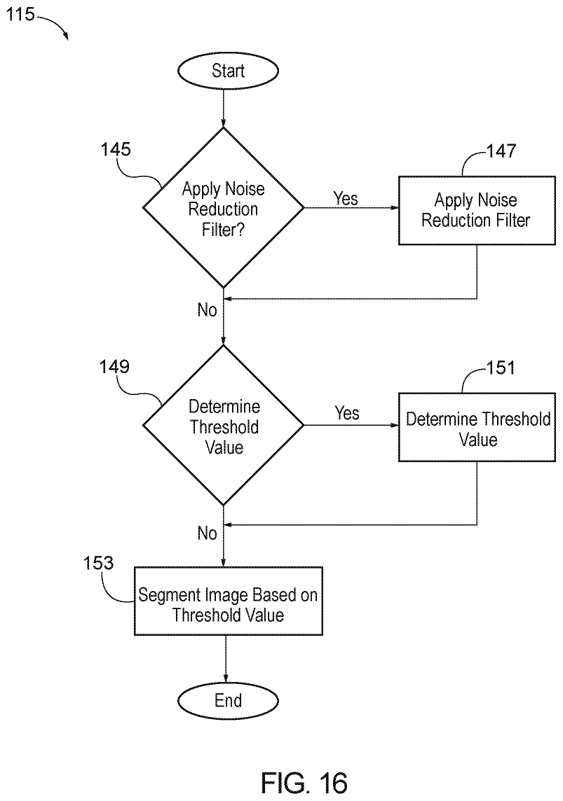

As shown in FIG. 16, some versions of image segmentation step 115 may begin with a step 145. In step 145, a determination is made regarding whether to apply a noise reduction filter to enhanced image 121. If step 145 determines a noise reduction filter should be applied, step 145 proceeds to a step 147 where the noise reduction filter is applied. Step 147 thereafter proceeds to a step 149. If step 145 determines that a noise reduction filter should not be applied, step 145 proceeds directly to step 149. In step 149, a decision is made regarding whether to dynamically determine a threshold value. If step 149 decides a threshold value should be dynamically determined, step 149 proceeds to a step 151 where the threshold value is determined. Step 149 thereafter proceeds to a step 153. If step 149 decides not to dynamically determine a threshold value, a static pre-determined threshold value is used and step 149 proceeds directly to step 153. In step 153, enhanced image 121 is segmented using the selected threshold value and step 153 and thereafter image segmentation step 115 proceeds to end.

Once image segmentation step 115 generates segmented image 143, image segmentation step 115 proceeds to data extraction step 117. In data extraction step 117, the background and foreground pixels are considered to derive information, such as the number of microbes in the sample. In some versions of data extraction step 117, the actual microbe count is compared with an average microbe count to determine if an error occurred within the image capture process. The comparison may incorporate a standard deviation with the average microbe count to generalize the microbe comparison.

Data extraction step 117 may be configured to derive information regarding the number of microbes in the image. In some embodiments of data extraction step 117, the number of foreground pixels in segmented image 143 may be counted in accordance with a predefined width and/or length to determine the number of microbes in the imaged portion of the inoculum. The counting algorithm may be divided into two separate algorithms, one for counting rod shaped microbes and one for counting spherical shaped microbes as the profile of the underlying microbes provides a corresponding different foreground pixel shape in segmented image 143. For example, the counting algorithm may be configured to consider a square of 2×2 pixels a microbe for counting purposes for spherical shaped microbes, or may consider a rectangle of 1×4 pixels a microbe for counting purposes for rod shaped microbes. Further, the counting algorithm may be configured to process both algorithms in order to capture the different three-dimensional orientations of rod shaped microbes. For example, if an elongated rod is positioned endwise towards AST camera 35, it will have a much different profile when viewed in two dimensions through AST camera 35. Therefore, both of the counting algorithms may be used during the counting phase of image analysis cycle 102. Alternatively, the counting algorithm may be configured to consider and count any foreground pixels surrounded by background pixels as a microbe.

Once data extraction step 117 derives the desired information from segmented image 143, image analysis cycle 102 terminates. Optimized AST method 101 iteratively performs image analysis cycle 102 at set time intervals to determine how the microbes in each test well 20 are changing and reacting to the particular antimicrobic dilution pairing. Further, optimized AST method 101 iteratively performs image analysis cycle 102 on each test well 20 associated with the microbes to determine how the microbes are reacting to each concentration of the antimicrobic dilution. For example, presume the microbes being tested are E. coli bacteria and three test wells 20 are being tested, with each test well 20 having a 20-microliter solution therein. The first test well 20 may contain an antimicrobic dilution of 1 microgram per milliliter (μg/ml), the second test well 20 may contain an antimicrobic dilution of 2 μg/ml, and the third test well 20 may contain an antimicrobic dilution of 4 μg/ml. Optimized AST method 101 performs image analysis cycle 102 on each of the three test wells at each set time interval to determine (a) how each antimicrobic dilution is affecting the microbes; and (b) how each antimicrobic dilution is performing relative to the other antimicrobic dilutions. If the data indicates the 1 μg/ml antimicrobic dilution is as effective as the 2 and 4 μg/ml antimicrobic dilutions at neutralizing the microbes, the 1 μg/ml antimicrobic dilution is the MIC.

An exemplary version of optimized AST method 101 is illustrated in FIG. 17 and begins with a step 155. In step 155, the system waits for a set time period threshold to allow the microbes within the selected test well 20 enough time to provide new information regarding the growth rate or reaction to the particular antimicrobic dilution. Optimized AST method 101 may preferably be configured to utilize a time period of 30 minutes for this threshold, though different thresholds may be used in different embodiments and/or with different microorganisms. For example, some bacteria or yeast or other microbes may react very quickly and provide information relevant to making an MIC determination within an hour. In this scenario, optimized AST method 101 may be configured to perform image analysis cycle 102 every five minutes to capture data regarding the rapidly changing environment within test wells 20. Other microbes may react relatively slowly to antimicrobic dilutions, and therefore a time period threshold of one hour may be more appropriate. Once step 155 waits the specified time period threshold, step 155 moves to a step 157.

In step 157, one iteration of image analysis cycle 102 is performed on a particular microbe with a selected test well 20. As discussed above, an iteration of image analysis cycle 102 derives data regarding the growth rate of the microbes within the selected test well 20. After an iteration of image analysis cycle 102 is performed, step 157 moves to a step 159. In step 159, the data collected in step 157 is stored and/or updated in memory, which may be in the form of a database, a flat file, or any other similar memory or storage device. In some embodiments of optimized AST method 101, step 159 stores the data collected in step 157 in database 71 (FIG. 6). Once step 159 stores/updates the collected data, step 159 moves to a step 161.

In step 161, optimized AST method 101 determines whether enough data has been collected to determine a MIC. This could be done, for example, by determining whether the data collected regarding the test well matched the data used to train a machine learning model used in determining a MIC and/or whether the machine learning model was able to make a determination with sufficient confidence to be usable. If more data is needed to accurately determine a MIC, step 161 returns to step 155 and waits to perform another image analysis cycle 102 to collect more data at a future time interval. If step 161 determines a sufficient amount of data has been collected, step 161 proceeds to a step 163.

The determination of whether the machine learning model is able to make a determination of an MIC with sufficient confidence to be usable can be based on a plurality of different criteria. A sufficient confidence value/level may be based on a threshold value, such that a useful determination may be considered to be when one or more of the determined MIC have a confidence value above the threshold value. The threshold value and the confidence value may both be expressed as a percentage value, e.g. 80%, 90%, 95%, etc., as a decimal representation, e.g. within a range between 0 and 1, or as any other suitable metric. The method may also determine that more data is needed to accurately determine an MIC if a plurality of confidence levels of a plurality of MIC determinations are within a specified confidence range of one another. In the example in which the confidence value is expressed as a percentage, the specified confidence range may be ±0.01% to ±5%. For example, if there are a plurality of MIC determinations having confidence values of 80%, 81% and 79%, which equates to a confidence range of ±2%, the method may determine that more data is needed to accurately determine the MIC for the biological sample.

In step 163, the MIC is determined. The determination is based on the data collected during each iteration of image analysis cycle 102 for all of the antimicrobic dilutions for a microbial sample as well as for a control well where no antimicrobial agent was present. As described in the following section, this determination may involve processing the collected data with a machine learning model that had previously been trained to make determinations of whether an antimicrobial dilution in a test well would inhibit growth and then determining the MIC based on the test well with the lowest concentration where the machine learning model made a determination that growth would be inhibited.

### III. RECURRENT NEURAL NETWORKS FOR DETERMINING MINIMUM INHIBITORY CONCENTRATION (MIC)

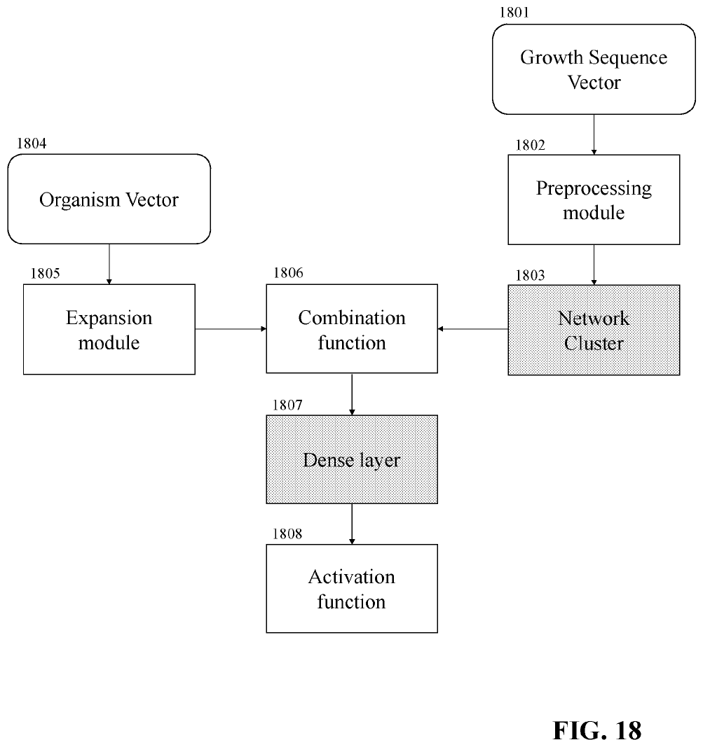

FIG. 18 depicts an exemplary architecture for a machine learning model that some embodiments may use to apply recurrent neural networks to the task of MIC identification. In an embodiment following FIG. 18, a growth sequence vector 1801 could be generated by periodically (e.g., every 30 minutes) capturing data from test wells and organizing it into a vector data structure. This growth sequence vector 1801 could then be provided to a preprocessing module 1802 to put the data from the growth sequence vector 1801 in a form more suitable for subsequent processing by other modules as shown in FIG. 18. For example, in some embodiments where subsequent processing is performed using linear equations, a preprocessing module could take raw count data and apply a log base 2 transformation to transform the raw data into a linear scale representing organism doublings. Other transformations may also be applied, either in addition to or as alternatives to the logarithmic transformation described above. For example, in some embodiments, differences between time step values could be calculated (e.g., to obtain figures for changes in doubling rather than simple organism doubling values).

The machine learning model may, therefore, receive, be provided with, or otherwise obtain a plurality of images, or image items, for each test mixture from a plurality of test mixtures, using the image capture techniques and methods described above, for example as shown in FIG. 8. The machine learning model may also receive, be provided with, or otherwise obtain a plurality of imaging times at which each of the plurality of images was captured. The plurality of imaging times may be used in conjunction with the plurality of images to recognize the growth patterns for microbes in the biological sample, and based on the training data provided to the machine learning model or otherwise, make growth rate predictions based on temporal sequences. The temporal sequences may be determined based on the time evolution, or rate of change of the growth rate, of the microbes as determined from the plurality of images and the plurality of imaging times for each test mixture of the plurality of test mixtures. The determination of a temporal sequence and the time evolution, or rate of change of the growth rate, of microbes within each test mixture of the plurality of test mixtures may be performed by any appropriate mathematical method or algorithm.

To illustrate, tables 1-4, below, provide examples of raw organism count data such as could be included in a growth sequence vector 1801, and how that count data could be transformed into doubling change values using a preprocessing module 1802 as described above.

TABLE 1
Exemplary organism count values for 0-180 minutes. Note
that each row represents a separate dilution, and therefore
a separate test well. In some embodiments, each row would
be represented as a separate growth sequence vector.

| | Organism Count at Time (min) | | | | | | |
|---|---|---|---|---|---|---|---|
| Dil. | 0 | 30 | 60 | 90 | 120 | 150 | 180 |
| 0 | 353 | 399 | 492 | 702 | 997 | 1579 | 2760 |
| 0.03 | 318 | 335 | 398 | 648 | 1006 | 1634 | 2938 |
| 0.06 | 462 | 508 | 567 | 774 | 1127 | 1707 | 3096 |
| 0.12 | 423 | 485 | 555 | 795 | 1218 | 1909 | 3467 |
| 0.25 | 88 | 110 | 205 | 450 | 872 | 1683 | 3341 |
| 0.5 | 73 | 135 | 274 | 578 | 1153 | 1935 | 3706 |
| 1 | 110 | 152 | 247 | 543 | 949 | 1832 | 3482 |
| 2 | 108 | 159 | 277 | 537 | 1038 | 1953 | 3748 |
| 4 | 85 | 123 | 236 | 578 | 1024 | 1915 | 3785 |
| 8 | 150 | 214 | 318 | 576 | 992 | 1923 | 3619 |
| 16 | 171 | 200 | 321 | 573 | 941 | 1838 | 3391 |
| 32 | 78 | 104 | 258 | 525 | 1004 | 2003 | 4000 |
| 64 | 79 | 130 | 244 | 520 | 953 | 1809 | 3610 |

TABLE 2
Exemplary organism count values for 180-360 minutes. Note
that each row represents a separate dilution, and therefore
a separate test well. In some embodiments, each row would
be represented as a separate growth sequence vector.

| | Organism Count at Time (min) | | | | | | |
|---|---|---|---|---|---|---|---|
| Dil. | 180 | 210 | 240 | 270 | 300 | 330 | 360 |
| 0 | 2760 | 5584 | 10467 | 18537 | 12825 | 36344 | 52218 |
| 0.03 | 2938 | 5511 | 10181 | 18794 | 14816 | 32115 | 52049 |
| 0.06 | 3096 | 5726 | 10567 | 18860 | 12954 | 39873 | 53187 |
| 0.12 | 3467 | 6488 | 11622 | 19689 | 19270 | 42501 | 53789 |
| 0.25 | 3341 | 6425 | 11516 | 20083 | 29713 | 41704 | 52317 |
| 0.5 | 3706 | 6832 | 12168 | 20542 | 26155 | 38117 | 45896 |
| 1 | 3482 | 5976 | 8081 | 9048 | 9201 | 8307 | 7183 |
| 2 | 3748 | 5816 | 7342 | 7782 | 7197 | 6391 | 5311 |
| 4 | 3785 | 6001 | 7557 | 7655 | 7241 | 6554 | 5172 |
| 8 | 3619 | 5702 | 6859 | 7022 | 6524 | 5677 | 4603 |
| 16 | 3391 | 5336 | 6433 | 6603 | 6306 | 5328 | 4378 |
| 32 | 4000 | 6262 | 7343 | 7294 | 7081 | 6059 | 5149 |
| 64 | 3610 | 5357 | 6516 | 6640 | 6308 | 5489 | 4638 |

TABLE 3
Exemplary organism doubling change values that could be provided
by a preprocessing module based on the count values of table 1.

| | Change observed at time (min) relative | | | | | |
|---|---|---|---|---|---|---|
| | to most recent doubling value | | | | | |
| Dil. | 30 | 60 | 90 | 120 | 150 | 180 |
| 0 | 0.1767 | 0.3023 | 0.5128 | 0.5061 | 0.6633 | 0.8057 |
| 0.03 | 0.0751 | 0.2486 | 0.7032 | 0.6346 | 0.6998 | 0.8464 |
| 0.06 | 0.1369 | 0.1585 | 0.4490 | 0.5421 | 0.5990 | 0.8589 |
| 0.12 | 0.1973 | 0.1945 | 0.5185 | 0.6155 | 0.6483 | 0.8609 |
| 0.25 | 0.3219 | 0.8981 | 1.1343 | 0.9544 | 0.9486 | 0.9892 |
| 0.5 | 0.8870 | 1.0212 | 1.0769 | 0.9963 | 0.7469 | 0.9375 |
| 1 | 0.4666 | 0.7004 | 1.1364 | 0.8055 | 0.9489 | 0.9265 |
| 2 | 0.5580 | 0.8009 | 0.9550 | 0.9508 | 0.9119 | 0.9404 |
| 4 | 0.5331 | 0.9401 | 1.2923 | 0.8251 | 0.9031 | 0.9829 |
| 8 | 0.5126 | 0.5714 | 0.8570 | 0.7843 | 0.9549 | 0.9122 |
| 16 | 0.2260 | 0.6826 | 0.8360 | 0.7157 | 0.9659 | 0.8836 |
| 32 | 0.4150 | 1.3108 | 1.0249 | 0.9354 | 0.9964 | 0.9978 |
| 64 | 0.7186 | 0.9084 | 1.0916 | 0.8740 | 0.9246 | 0.9968 |

TABLE 4
Exemplary organism doubling change values that could be provided
by a preprocessing module based on the count values of table 2.

| | Change observed at time (min) relative | | | | | |
|---|---|---|---|---|---|---|
| | to most recent doubling value | | | | | |
| Dil. | 210 | 240 | 270 | 300 | 330 | 360 |
| 0 | 1.0166 | 0.9065 | 0.8246 | −0.5314 | 1.5028 | 0.5228 |
| 0.03 | 0.9075 | 0.8855 | 0.8844 | −0.3431 | 1.1161 | 0.6966 |
| 0.06 | 0.8871 | 0.8840 | 0.8358 | −0.5419 | 1.6220 | 0.4157 |
| 0.12 | 0.9041 | 0.8410 | 0.7605 | −0.0310 | 1.1411 | 0.3398 |
| 0.25 | 0.9434 | 0.8419 | 0.8023 | 0.5651 | 0.4891 | 0.3271 |
| 0.5 | 0.8824 | 0.8327 | 0.7555 | 0.3485 | 0.5433 | 0.2679 |
| 1 | 0.7793 | 0.4354 | 0.1631 | 0.0242 | −0.1475 | −0.2097 |
| 2 | 0.6339 | 0.3361 | 0.0840 | −0.1127 | −0.1714 | −0.2671 |
| 4 | 0.6649 | 0.3326 | 0.0186 | −0.0802 | −0.1438 | −0.3417 |
| 8 | 0.6559 | 0.2665 | 0.0339 | −0.1061 | −0.2006 | −0.3026 |
| 16 | 0.6540 | 0.2697 | 0.0376 | −0.0664 | −0.2431 | −0.2833 |
| 32 | 0.6466 | 0.2297 | −0.0097 | −0.0428 | −0.2249 | −0.2348 |
| 64 | 0.5694 | 0.2826 | 0.0272 | −0.0740 | −0.2006 | −0.2430 |

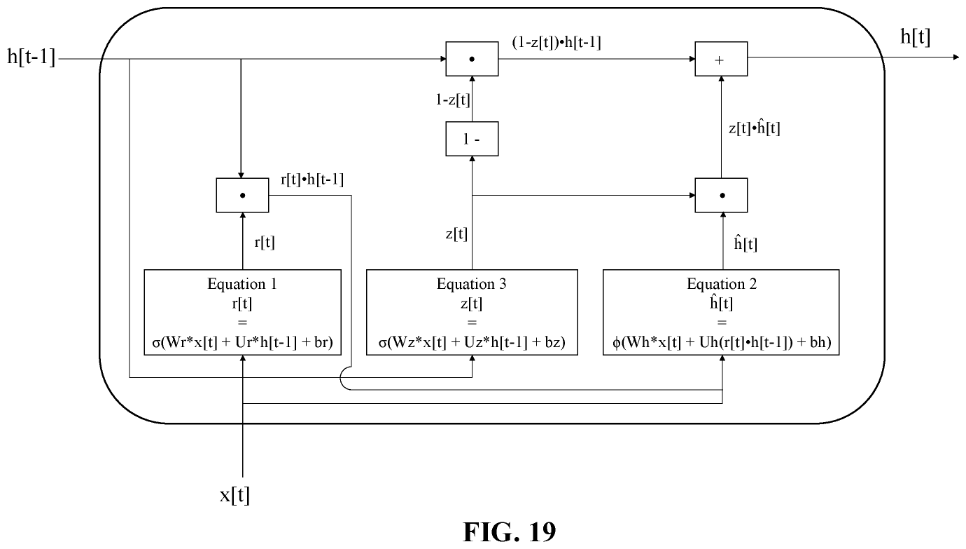

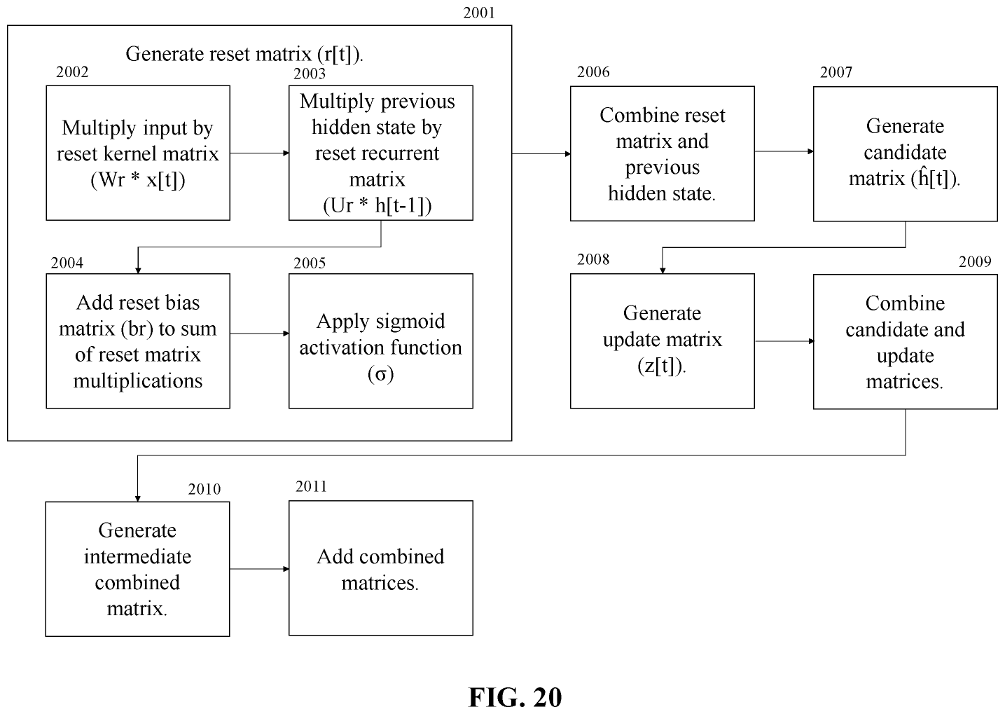

In an embodiment following the architecture of FIG. 18, after preprocessing was complete, the output of the preprocessing module 1802 would be provided to a network cluster 1803, e.g., as a series of 1×1 matrices representing the values obtained at individual time steps from the preprocessing calculations described above. This network cluster 1803 would preferably comprise a plurality of recurrent neural networks, such as long short-term memory (LSTM) or gated recurrent unit (GRU) networks. In an embodiment following the architecture of FIG. 18, such recurrent neural networks can be trained to recognize patterns which are relevant to specific organisms based on a dense layer 1807 identifying certain recurrent networks as better or worse for specific organisms. This would result in the dense layer 1807 up or down-weighting those recurrent neural networks accordingly, which would, in turn, cause them to become more effective at identifying patterns associated with their particular organisms through the training process. An illustration of a unit which could be trained in this manner and which could be included in a network cluster 1803, in this case a GRU, is provided in FIG. 19, and the calculations it would perform are discussed in the context of FIG. 20 and equations 1-4.

Turning now to FIGS. 19 and 20, FIG. 19 illustrates an individual GRU that could be used to recognize a particular growth pattern in the network cluster 1803 of an embodiment following the architecture of FIG. 18, while FIG. 20 illustrates in flowchart form the processing that such a GRU would perform. As shown in FIG. 20, at each time step (e.g., for every 30 minute data point) a reset matrix (r[t]) would be generated 2001 to specify information from previous time steps to discard or downweight. This can be done using Equation 1, which represents mathematically the substeps 2002 2013 2014 2005 shown in flow chart form as making up the reset matrix generation 2001 of FIG. 20.

r[t]=σ(Wr*x[t]+Ur*h[t−1]+br) Equation 1:

In those substeps, the input for a particular time step (x[t], where t is the time step in question) is multiplied 2002 by a reset kernel matrix (Wr) which weights the input to determine how much of the information carried over from the previous step (h[t−1]) should be forgotten. A similar multiplication 2003 is performed using a hidden state (h[t−1]) carried over from the previous time step (which, on the first time step, could be simply set to zeros), multiplying that hidden state by a reset recurrent matrix (Ur) which weights each of the items in the hidden state to determine the information from the previous step that should be forgotten. The results of these multiplications would then be added 2004 to a reset bias matrix (br) to obtain a raw reset matrix. This raw reset matrix would then be provided as input to a sigmoid activation function 2005 which would normalize its values by mapping them onto the range [0, 1], thereby providing the reset matrix (r[t]) which would be used to decide what (and how much) information from previous steps to discard.

Once it had been generated, the reset matrix r[t] could be combined 2006 with the hidden state from the previous time step (h[t−1]), such as by taking the Hadamard product (represented in FIG. 19 and Equations 2 and 4 by ·) of the reset matrix and the hidden state, this could then be used, along with the input for the then current time step (x[t]), to generate 2007 a candidate matrix (ĥ[t]) which could be used to update the state value of the GRU. In some embodiments, this type of generation 2007 could comprise application of Equation 2.

ĥ[t]=ϕ(Wh*x[t]+Uh(r[t]·h[t−1])+bh) Equation 2:

In that equation, ϕ is a hyperbolic tangent function which maps the output onto [−1, 1], and Wh, Uh and bh are candidate kernel, recurrent and bias matrices which would be applied in a manner similar to that described for the reset kernel, recurrent and bias matrices used to generate 2001 the reset matrix (r[t]).

Once the candidate matrix (ĥ[t] had been generated 2007, an embodiment following FIG. 20 would also generate 2008 an update matrix (z[t]) using Equation 3 and update kernel, recurrent and bias matrices (respectively, Wz, Uz, and bz) in a manner similar to that described above for the generation 2001 of the reset matrix using Equation 1.

z[t]=σ(Wz*x[t]+Uz*h[t−1]+bz) Equation 3:

This update matrix (z[t]) could then be combined 2009 with the candidate matrix (h[t]) in a manner similar to that used to combine the reset matrix and the hidden state from the previous step (e.g., taking the Hadamard product). An additional intermediate combined matrix could then be generated 2010 by subtracting the update matrix (z[t]) from 1, and combining it with the hidden state matrix from the previous step (h[t−1]). These two combined matrices could then be added 2011 together to provide a final matrix (h[t]), which could be expressed mathematically as the result of Equation 4.

h[t]=(1−z[t])·h[t−1]+z[t]h[t] Equation 4:

This final matrix (h[t]) could serve as both the output of the GRU at time step t, as well as the hidden state that could propagate forward to the next time step. In this way, embodiments utilizing a GRU such as shown in FIG. 19 could take advantage of the ability of recurrent neural networks to identify patterns over time to make more accurate determinations of whether the particular concentration and antimicrobial in a particular test well would inhibit microbial growth.

After the GRUs (or other types of networks) from the network cluster 1803 had processed the output of the preprocessing module 1802, the result would preferably be a set of 1×1 matrices, with one matrix being provided by each unit in the network cluster 1803 (e.g., if the network cluster 1803 consisted of 24 GRUs, then the output of the network cluster 1803 would be 24 1×1 matrices). This set of matrices could then be combined with a vector representing the organism for which the MIC determination was being made. As shown in FIG. 18, information on an organism could follow a different path than that described for the growth sequence vector. For example, in some embodiments, a machine learning model following the architecture of FIG. 18 could be adapted to make MIC determinations for a plurality or organisms, and each of those organisms could be given a particular identification using one hot coding. For instance, in a model adapted to make MIC predictions for four organisms, the first could be given the ID [1 0 0 0], the second could be given the ID [0 1 0 0], the third could be given the ID [0 0 1 0], and the fourth could be given the ID [0 0 0 1]. When using a model following the architecture of FIG. 18, an organism vector 1804 with information identifying the organism for which MIC is being identified could be provided as an input to an expansion module 1805, which could transform the organism vector 1804 into a form compatible with the output of the network cluster 1803. For example, in an embodiment where the output of the network cluster 1803 consisted of a set of 24 matrices, the expansion module could duplicate the organism vector 24 times to create a data structure which included one vector for each of the output matrices.

After the organism vector 1804 had been put into a form compatible with the output of the network cluster 1803, the outputs of the expansion module 1805 and the network cluster 1803 could be provided to a combination function 1806. This combination function 1806 could then join them, such as by multiplication or concatenation. The combined output could then be provided to a dense layer 1807, which, in some embodiments, could be implemented as a linear equation that applies weights to the output of the combination function. In embodiments using this type of approach, the portions of the combined output that are populated by the output of the network cluster 1803 could be seen as providing measures of growth, while the portions of the combined output that are populated by the output of the expansion module could be seen as acting as a mechanism for applying a different bias based on the organism in question.

Once it had been calculated, the output of the dense layer 1807 would be provided to an activation function 1808 for normalization (e.g., a sigmoid activation function), and the output of the activation function 1808 could be seen as a determination, based on the information gathered as of the time step that the growth sequence vector 1801 was provided as input, of whether the antimicrobial concentration of test well from which the growth information was gathered would successfully inhibit growth of the microorganism. For example, in the case of a sigmoid activation function, a value of 1 could be interpreted as a determination that growth would not be inhibited, a value of 0 could be interpreted as a determination that growth would be inhibited, and values between 0 and 1 could be treated as determinations of growth or no growth at confidence levels based on how close the value was to 0 or 1. This could then be compared with determinations of growth or no growth for the other test wells, and lowest concentration of antimicrobial in a test well with a determination of no growth could be treated as the MIC. Additionally, in some embodiments, an additional bias value (referred to as a dense bias) may be added to the output of the dense layer 1807 to cause the activation function 1808 to be triggered by different types of outputs, thereby allowing for further tuning.

Of course, it should be understood that, while some embodiments may use an architecture as shown in FIG. 19 for determining MIC, variations are also possible and may be implemented by those of ordinary skill in the art. For example, while some embodiments may include a network cluster 1803 which outputs a 1×1 matrix or single scalar value for each unit making up the cluster, it is also possible that a network cluster 1803 may provide output in the form of a set of scalar values/1×1 matrices for each unit, where the set of scalar values/1×1 matrices includes one value for each time step value from the growth sequence vector 1801. In such an embodiment, the dense layer 1807 may include a weight for each of the scalar values/1×1 matrices for each of the units from the network cluster 1803, thereby allowing for more information to be considered in making the determination of whether the concentration of antimicrobial in a particular test well would or would not inhibit microorganism growth. Other variations on the dense layer 1807 are also possible, and may be made independent of the outputs of the network cluster 1803. For example, while the discussion of FIG. 18 referred to embodiments where the dense layer 1807 is implemented as a linear equation that applies weights to the output of the combination function, in other embodiments, a dense layer 1807 could be implemented in other manners, such as a multi-layer neural network with input nodes for each value provided by the combination function and one or more hidden layers through which those input notes would be connected to an output activation function. Accordingly, the above description of variations should be understood as being illustrative only, and should not be treated as limiting.

As another example of a type of variation that could be implemented by those of ordinary skill in the art, in some embodiments multiple instances of models using an architecture as shown in FIG. 18 could be used in determining MIC. For example, in some cases there could be multiple instances where each instance had different trainable layers (e.g., network clusters made up of LSTMs rather than GRUs, network clusters and/or dense layers trained with a bias toward predicting growth or no growth, network clusters with different numbers of units such as 16 or 32 rather than 24, etc.). In such embodiments, the multiple instances could be applied in parallel at each time step, and the determination of growth or no growth could be made using a voting protocol which would allow low confidence determinations from individual instances to be combined to make an earlier determination than would be possible using any of the individual instances on its own. As another example of how multiple instances of models using an architecture such as shown in FIG. 18 could be used, some embodiments may include multiple instances where each instance was trained to make a determination at a particular time step. For example, there could be one instance trained to make a determination four hours after inoculation, one instance trained to make a determination five hours after inoculation, and one instance trained to make a determination six hours after inoculation. In such an embodiment, if an instance was not able to make a determination with sufficient confidence, then the data collection process could continue until enough data had been gathered for a determination by the next instance, and this could then be repeated until ultimately a sufficiently confident determination could be made.

Variations are also possible in how a model following the architecture of FIG. 18 could be trained. For example, while it is possible that the trainable portions of a model following the architecture of FIG. 18 (set off using grey fill for ease of reference) could be trained using standard feedback and reinforcement techniques, it is also possible that some modifications could be made to facilitate the training. For instance, in some embodiments, an architecture such as shown in FIG. 18 could be modified for training purposes by inserting a filtering layer between the network cluster 1803 and the combination function 1806. Such a filtering layer could reduce the amount of data passed on from the network cluster 1803 (e.g., if the network cluster made a MIC determination for time steps separate from each other by 30 minutes, a filtering layer could pass on only the determinations made every hour to the combination function 1806). As another example, while some embodiments may perform training only on final outputs of a model (e.g., if the model was being trained to make a MIC determination after 6 hours, then the training would be on whether the determination made at 6 hours was correct), other embodiments may compare outputs at each time step with target outputs provided by the training data, so that a loss function and appropriate updating could be performed for all time steps where training data was available. Other variations are also possible and will be immediately apparent to those of ordinary skill in the art in light of this disclosure. Accordingly, the above description of training variations should be understood as being illustrative only, and should not be treated as limiting.

As an illustration of how these types of training approaches could be applied in practice, consider tables 5-12, below. In those tables, tables 5-9 provides GRU kernel weights, GRU recurrent weights, GRU bias weights, dense kernel weights, and a dense bias weight for a model implemented using the Keras API (an open source project governed by the Keras Special Interest Group and available at https://keras.io) and Tensorflow backend (an open source machine learning platform developed by Google, Inc. and available at https://www.tensorflow.org/) comprising a network cluster with 24 GRUs trained to make MIC determinations at 4, 5 and 6 hours based on training data for 56 microorganism strains when combined with various antimicrobials and the various combinations were labeled with growth or no growth information determined after 16 hours. In particular, table 5 provides a 1×72 array, in which the first 24 values would be mapped to Wz, the second 24 values would be mapped to Wr and the third 24 values would be mapped to Wh in an embodiment following FIG. 19. Table 6 provides a 72×24 array, in which the first 24×24 array (i.e., the first 24 bracketed sets of values) would be mapped to Uz, the next 24×24 array would be mapped to Ur and the third 24×24 array would be mapped to Uh in an embodiment following FIG. 19. Table 7 provides a 1×72 array, in which the first 24 values would be mapped to bz, the second 24 values would be mapped to br and the third 24 values would be mapped to bh in an embodiment following FIG. 19. Table 8 provides a 1×1344 array of values which would be multiplied by the output of the combination function 1806 in the dense layer 1807. Table 9 provides a bias value which would be added to the output of the dense layer 1807 before it was provided to the activation function 1808. Table 10 provides growth/no growth determinations made at 4, 5 and 6 hours by a system trained as described above for Pseudomonas aeruginosa and the drug Ceftazidime. Tables 11-12 provide microorganisms and antimicrobials included in the training data described above and for which the model was confirmed to be effective.

TABLE 5
Exemplary GRU Kernel Weights

[−0.66256541 0.80381989 0.18491858 −0.54941893 −0.03170178 0.15720482
0.06896526 −1.58856416 1.65793538 0.80985379 0.33203655 −1.77530289
−1.76799452 −0.23928253 0.1523208 0.42538524 −1.13591826 −0.03369806
−1.18132961 −0.96825945 0.11378209 −0.66428781 −0.49829632 −0.61977792
0.35420355 −0.00912795 0.64135551 0.16730106 −1.11411262 1.61123979
0.5604741 −0.79450673 0.50121385 0.97526222 0.77369064 −1.63763225
−1.57868218 0.22748969 1.36069262 1.19656217 0.64924759 −0.51427883
2.77376199 0.25395295 1.1319567 −1.43182874 −2.16484165 0.96999162
−0.29644144 −2.14624596 1.01429033 −0.14247622 −0.20825356 0.72008324
−0.894692 −0.71999758 −2.75401711 −1.26149702 −0.46498731 1.73494327
−0.81529969 −0.1448967 0.80373144 1.40460062 −0.60051787 −0.17547813
0.03729261 −1.16215646 0.5213111 2.30003786 −0.7754879 −0.04352728]

TABLE 6
Exemplary GRU Recurrent Weights

[−0.25234213 −2.06746435 0.20451522 −0.60107684 2.42755508 −0.90039349
−0.23871075 0.21430588 −0.48221892 −1.83985639 0.06906345 −0.6850509
1.19502759 0.78425741 −0.61191905 −0.77736157 −0.02499971 0.24460767
0.11608241 −0.75061792 −3.21089172 −1.09927428 0.19700123 0.95441514
0.61443663 0.10292193 0.45450923 −0.29507631 −0.96234256 0.04782755
−0.09688304 −1.11779726 −0.56428319 −0.31762978 −0.1721586 −1.31495678
0.45842746 0.85037875 −0.26819858 0.21549052 −0.20583533 −0.38104945
0.51194555 −0.40792108 −0.55547631 −0.62908578 0.04660884 0.87944245
1.79305017 −0.21559033 −0.10059903 0.02641164 −0.726412 0.14513539
−0.59741735 0.01661355 0.14167534 0.17504099 0.14853439 0.05526508
−1.99298453 0.08702543 −0.99891728 0.64159542 −0.26831791 1.16208434
−0.01835633 0.23698957 0.16372122 0.13721746 0.39733502 0.14299327]
[−2.12296724e−01 1.88613296e−01 −1.36501417e−01 3.63255799e−01
6.76352561e−01 −5.48835635e−01 −1.43678501e−01 3.03257847e+00
4.66163814e−01 −1.67858958e−01 6.11010909e−01 3.55418491e+00
1.97571301e+00 9.11602855e−01 3.22472632e−01 2.97426376e−02
−5.16661704e−01 9.86973226e−01 4.71715033e−01 8.21583495e−02
1.15462869e−01 7.15637624e−01 1.03700566e+00 4.39825624e−01
−2.55649418e−01 −6.72942281e−01 −1.83586150e−01 9.05950144e−02
−7.12451696e−01 −1.61050528e−01 3.03788990e−01 −5.66104293e−01
1.19426094e−01 −8.83606851e−01 −4.81469631e−01 −1.74526170e−01
6.04901969e−01 1.29873559e−01 3.64972293e−01 3.16877663e−03
1.90621212e−01 −2.36938739e+00 3.57759267e−01 4.43094134e−01
2.36005113e−02 −4.60000098e−01 2.19438270e−01 5.81247687e−01
1.09162581e+00 −3.54853600e−01 2.95531034e−01 3.67801040e−01
−9.96347964e−01 −6.40432537e−01 1.00378923e−01 1.93714008e−01
−3.56398135e−01 7.41557121e−01 1.11293569e−01 −4.16331261e−01
−1.68146360e+00 −3.43616724e−01 −4.36586291e−01 −1.29879504e−01
3.86749685e−01 −5.78480721e−01 8.34003866e−01 4.02766943e−01
−3.41357961e−02 1.26936594e−02 −1.43037367e+00 6.99962080e−01]
[−0.27206773 −0.50782067 −0.51656836 −0.66009307 −0.08239018 −0.023389
0.1149777 −0.88448381 0.62018853 −0.32238552 1.98147547 −0.66136122
0.17299929 0.51408714 −0.32199198 −0.72598612 0.58763647 0.0944607
0.1388377 −0.32068342 −1.118644 −0.52827531 −0.99434346 0.41150409
0.38826072 0.0920589 −0.78346664 0.06319764 0.6304161 −0.39609781
−0.72676742 0.15888509 −0.56240451 0.03219106 −1.97552419 0.08964109
−0.43053648 0.17825276 −0.06717906 0.17368394 −0.15334943 −0.30977064
−0.14668143 −0.40658253 −0.24039008 0.07762149 −0.08916742 −0.63524079
0.31993324 −0.41415295 1.04928958 −0.37737018 0.7936067 −0.24234152
−0.52914423 0.52732432 −0.08318374 −0.01055499 −0.31334716 −1.09639227
0.08035888 −0.56494886 −0.31433147 −0.74616295 −0.73096925 0.25502339
0.30952862 −0.39934391 0.88490653 0.13033056 1.08081508 0.51069385]
[2.55640239e−01 −7.88450181e−01 −2.46783972e−01 4.50114876e−01
3.37848663e−01 −5.79967737e−01 1.13276315e+00 −7.35817552e−01
2.80036658e−01 −6.56879425e−01 5.12681305e−01 5.38315415e−01
−6.36223674e−01 3.12631130e−01 1.63657427e−01 −2.70610809e−01
7.59213448e−01 1.55449018e−01 4.45806682e−02 2.74026603e−01
−8.94355357e−01 −9.39703524e−01 −2.51555651e−01 3.76930714e−01
3.71809453e−01 1.55255437e−01 1.17686510e−01 −5.81000507e−01
−7.02717245e−01 −5.81550896e−01 −6.05461597e−01 −5.65197825e−01
7.60254920e−01 −3.37900251e−01 −2.23849987e−04 −1.66852683e−01
4.86888945e−01 2.02563539e−01 −6.82664812e−01 −9.44509566e−01
−7.50670195e−01 4.36588913e−01 2.08347037e−01 6.17936969e−01
6.46044135e−01 1.01910334e−03 4.59635645e−01 −6.20812252e−02
7.26950824e−01 −1.91848785e−01 6.79159939e−01 7.86851406e−01
−2.12426414e−03 5.71344554e−01 1.95132911e+00 3.90225649e−01
−6.20344095e−02 −1.30451992e−01 −5.15717566e−01 −7.76126206e−01
−6.13328397e−01 −4.13317800e−01 −1.01301265e+00 −6.49802327e−01
3.22740853e−01 5.49745440e−01 −6.14329219e−01 1.70654774e−01
5.45662701e−01 −2.53557861e−01 4.99824971e−01 3.70813860e−03]
[−6.05572224e−01 −5.17917216e−01 −1.01257813e+00 −1.28841370e−01
−4.62006956e−01 4.90625978e−01 −1.21877062e+00 −2.82967782e+00
4.62933838e−01 −3.38539898e−01 5.19617736e−01 −1.32296669e+00
−5.27978122e−01 3.09180558e−01 −5.09667993e−01 −2.15177342e−01
3.95683162e−02 4.60725963e−01 3.45500022e−01 −2.51403284e+00
2.91572779e−01 −6.94102645e−01 −3.57299566e−01 2.67681420e−01
−2.34981418e−01 1.42265096e−01 −5.92432797e−01 −2.81693786e−01
6.23638451e−01 −6.29889518e−02 −2.67232537e−01 −5.48590600e−01
4.70704725e−03 −1.36510819e−01 4.36327875e−01 −2.26700038e−01
1.14936665e−01 1.12044394e+00 1.45568907e−01 −1.00230679e−01
2.71748096e−01 1.47411576e−03 3.87936026e−01 −4.93935466e−01
1.80674300e−01 −3.39481421e−02 −8.03405941e−02 1.02844393e+00
3.17578375e−01 7.44189844e−02 −1.49251604e+00 3.60372484e−01
2.63372093e−01 1.53689966e−01 6.36319101e−01 6.01285517e−01
9.31313932e−02 5.81428334e−02 −1.35313094e+00 −5.98998606e−01
9.83568311e−01 −1.74461269e+00 5.35590434e−03 −1.61683828e−01
3.18641394e−01 −1.10580496e−01 5.74967451e−03 1.08825898e+00
−8.28042328e−01 −5.16686976e−01 1.28213835e+00 −4.23299074e−01]
[0.16143046 −0.31814429 −0.20373066 −0.09196647 0.18693213 0.54654771
−0.06970128 −0.87548828 0.49112746 −0.5853315 0.5188027 −2.40321136
−0.60974526 0.25555933 −0.20026587 −0.27759886 0.76192319 0.36541089
0.46618044 −0.60214293 0.13084359 −1.12116432 0.2444436 0.0819495
0.41971314 0.02356632 0.10584333 0.2407898 0.38873059 0.10266422
0.11688064 0.18508729 0.69825888 0.19438772 0.06760285 −0.02760804
−0.06694215 1.04355085 0.1707949 −0.11045007 −0.03260897 0.05721386
0.40264639 −0.46487224 0.12793361 0.42832258 0.01040957 0.71740854
−0.0526942 −0.09922143 0.62718987 0.2059831 1.22861743 −0.11428503
−0.03132821 0.02312227 −0.68112636 −0.3113409 −0.49964577 0.04008607
0.61901474 −0.79387903 0.09821174 0.16391483 −0.28234047 −0.2136149
−0.35124919 −0.25075161 0.11601611 0.01200502 1.16455877 −0.33065194]
[0.35341594 −0.65655613 −0.16044892 0.37403727 1.7585659 −0.76756585
0.36583528 −0.01325926 −0.13395593 −0.9676137 0.21529181 −0.17655726
0.1687464 0.19650242 −0.42987385 −0.38571021 −0.2895515 −1.67001295
−0.13010517 0.61078042 −1.72121978 −1.90940344 −0.094869 0.40947407
0.31696963 0.02292828 0.91065198 −1.31932795 −0.38892445 −0.2769928
−0.7420001 −1.56817269 1.01365614 −0.32538626 0.48438373 −0.46527892
0.5567857 0.27826491 −0.81795686 −0.93899107 −0.00510397 −0.06708363
0.25813678 0.46574855 0.43622047 −0.13971618 −0.1596799 0.41583723
1.24780929 −0.02720818 0.16151313 0.04021943 −0.97094679 0.77095044
0.78781068 1.32333219 0.09552483 −0.48921832 −0.45301443 −0.45986718
−0.97162104 0.16272107 −0.09900572 −0.36591044 0.91766453 −0.65769315
−0.41547433 0.25193894 0.62340313 0.0054449 0.38310716 0.26010442]
[−1.68168294 −0.76385117 −0.75764555 −0.3806909 1.37009633 −0.76501256
−0.09369998 0.03219106 −1.01101851 −0.48646769 −0.86331391 −0.53510332
−0.30326054 0.0307204 −0.51740825 −0.68689042 −2.04929209 −0.57708764
−0.63009453 −0.94928932 −0.56710911 −2.14315462 −0.20205308 0.69341952
0.42107102 0.10698017 1.16352975 −0.52162391 −1.21016204 −0.0247065
−0.04023665 0.15146455 0.61459899 0.11458327 0.13456234 −0.04463924
0.21400668 0.46792969 −0.45231426 −0.2044161 0.19264042 0.14629883
0.06707554 −0.27989203 −0.17711464 0.36058277 0.30169317 0.80415255
0.96640509 0.36006117 0.08280902 −0.38133344 −1.47433841 0.39198115
−0.05415284 0.87043637 0.3656528 −1.0819 −0.22308743 −0.34308994
−0.10558148 −0.43666738 0.09442204 −0.19245814 −0.14870043 0.95879179
−0.28299129 0.47304118 −0.75319135 0.52341932 −0.5476405 −0.02133688]
[0.46133906 0.54202783 0.59548712 −0.47788712 −0.44030911 0.23560879
−1.16246653 −0.0017727 1.31995821 −0.76756054 −0.21685326 1.48808336
1.48119962 0.09443004 −0.23001622 −0.9802323 −0.5789634 −0.25468647
−0.14122175 −0.07264496 0.63367462 −0.15167034 0.09934241 0.17697531
−0.71254241 0.28255108 1.18986535 0.48383945 −0.15752724 −0.57995141
−0.15782875 −0.30505303 0.18095925 0.16454916 0.71966243 0.27498585
0.41545352 0.72151899 −0.08498495 −0.22456704 −0.24328978 −0.3996256
0.66465819 0.30307889 −0.140962 0.12643373 0.04975802 1.16298532
0.59068745 0.53822279 −0.13896826 1.26442456 −0.12931035 0.13284251
0.18315274 −0.45165735 0.82412249 0.57367009 0.43832675 0.49805596
−0.05777021 −0.32562369 −0.50769031 0.23821612 0.67723435 0.97021484
0.17715172 0.49825573 −1.44080639 −0.17286213 0.06410505 −0.29090542]
[−3.62361193e−01 −2.47268811e−01 −2.49642968e−01 7.75110304e−01
−7.29234636e−01 3.79733741e−01 −4.83325347e−02 2.09953141e+00
−2.17473671e−01 −4.13605161e−02 −8.84272829e−02 5.47548294e−01
3.37701893e+00 −2.54765272e−01 1.13391697e+00 3.99736762e−01
−3.32802504e−01 3.14333528e−01 1.20832181e+00 4.02273655e−01
1.99296251e−01 4.25208956e−01 −3.80453348e−01 6.23716936e−02
−7.97254920e−01 −5.57713985e−01 5.08835375e−01 1.55576631e−01
−1.54122078e+00 −9.68651623e−02 −1.98469952e−01 −5.32426536e−01
3.47352624e−01 −1.65475816e−01 −1.38134718e−01 −6.34056628e−02
−1.52575538e−01 −7.45185494e−01 2.11715996e−02 −2.16128081e−01
3.01663607e−01 −1.19127893e+00 2.77991116e−01 −3.85425473e−03
7.98813224e−01 −4.09721099e−02 1.18905179e−01 −3.20858091e−01
3.62348825e−01 −3.53993118e−01 −4.48435068e−01 8.19005549e−01
−1.14332652e+00 3.47529292e−01 −3.08673177e−02 −1.70457959e−01
2.42676914e−01 5.60770810e−01 −8.21679384e−02 −1.46377578e−01
−2.27243876e+00 8.83481726e−02 −5.12931049e−01 −3.63863766e−01
5.43927066e−02 −3.28801125e−02 3.58798474e−01 5.28750896e−01
1.42743313e+00 −4.49691986e−04 −5.62742352e−01 1.42309725e−01]
[0.58208412 0.07730986 0.41285267 −0.10236336 −0.99875438 −1.20775819
0.44101745 2.19599175 −0.38262057 −0.2387694 −0.22580302 2.89699602
−0.38939089 −0.33241037 0.66092306 0.51522869 0.05869727 −0.01678781
−0.55306935 0.12563488 −0.55164135 0.58589184 0.85879111 −0.60101837
0.12361421 −0.14704831 0.84966469 −0.19688016 −0.25371993 −0.15962923
−0.19233386 0.16829775 −0.56479031 0.15465286 0.47215515 −0.24304165
−0.28758866 −1.7615273 −0.08674689 0.04042149 −0.37822655 0.29173532
−0.19059406 0.22525921 −0.04603248 0.37710363 0.13245195 −0.23565505
−0.45038289 −0.20984486 −1.13100839 −0.12375942 −1.12062347 0.21985675
0.1268248 −0.21998377 0.24359101 0.41163936 0.7407034 0.12217356
0.1620817 0.44563749 −0.30683398 −0.07190799 −0.12579985 0.17964041
0.27911192 −1.01916063 0.3582499 0.40211472 −1.23486292 0.17254248]
[1.44158399 0.50417095 0.80120474 −1.56288302 −1.5447309 0.31527913
0.98324668 −0.35315734 0.83516085 −0.05781088 −0.52371657 0.2531147
0.15336968 −1.00881791 1.49754584 1.42022526 1.12887192 0.29830348
−0.56812823 0.39573056 0.82045919 2.33831382 −1.23845673 −0.53548598
0.64968097 −0.75210124 −0.20569679 −0.25592381 0.987351 −0.05486193
0.6084671 0.11183418 0.05975453 0.06903413 0.30913824 −0.08153434
0.51637846 −0.15211831 1.07019091 0.71754092 −0.29677007 0.35256779
0.10056991 0.33086851 0.10437057 −0.08477195 0.10641313 0.17704685
−2.15175009 −0.45271912 0.6982438 −0.30008787 −0.34382135 0.19435579
0.11772324 −2.22127724 −0.33179918 0.37488624 0.44093248 −0.8028366
0.18721479 1.70294797 −0.07531694 0.97517562 −0.1550428 0.35455391
0.46210611 −0.63885856 0.00309271 −0.54106069 −0.35911885 −0.47542956]
[−0.02041281 −0.17677964 −0.30888984 0.19115819 1.0180217 0.26885521
−0.58074486 0.09289213 0.71674836 0.25793526 0.18387662 −1.17952859
−2.98410416 0.38896185 0.25660983 −0.02906585 0.11067195 0.18623361
−0.06486232 0.12189846 −0.22011229 −0.5111168 −0.63332444 −0.28034616
−0.0611867 0.52050364 −0.09660802 0.32067111 1.54999816 −0.48249152
0.19849664 0.27476948 0.38671452 0.14557569 0.43136528 −0.06660089
−0.14820333 1.22988462 −0.22385164 −0.045624 0.14418954 −0.57611912
−0.03114169 −0.07379089 −0.84169674 −0.35879576 0.04851702 −0.49067989
−0.12479126 0.12192555 1.1191256 −0.32489723 −0.45062491 −0.44508588
0.45537105 0.34179056 −1.34210169 0.02349343 0.11669461 −1.01926053
0.15449402 −0.4273679 0.2988967 −0.76890564 0.39498967 −1.8880204
0.46779498 1.03493428 −0.73144007 −0.57561213 0.26779929 0.07991653]
[−0.17775632 0.70672381 0.02738352 −0.13682722 −0.79407126 0.45290449
−0.49024248 −0.32830086 0.13880292 0.9037239 −0.38879338 1.46160364
0.26055256 0.2999312 0.01185164 −0.04889608 0.41444781 0.0370497
0.00641307 1.61027336 −0.28130436 0.79868835 1.08714092 −0.2136489
−0.03073524 −0.32157946 0.29025421 −0.16664356 −0.21213265 −0.20366117
0.07845494 −0.05328261 −0.34907579 −0.42540973 0.00251787 −0.06769898
−0.07807054 −0.99783128 −0.44038352 0.15602601 −0.11118671 0.00195016
−0.23832729 0.06717415 −0.86348081 0.12694596 0.17988899 −0.25563958
−0.56640846 0.22981898 −0.43209299 0.03084022 0.49875051 −0.32394418
0.17868927 −1.11377275 0.27838442 −0.07439093 1.13668239 0.260133
−0.29979548 0.84447592 0.06383254 −0.20362708 0.02923979 0.20106167
1.0655601 −1.44538331 −0.15993275 0.39271492 −1.61643267 1.45949912]
[−0.10566645 1.06520116 −0.02456725 −0.51630044 −1.59336948 0.83360386
0.08450241 −0.29233205 0.03279358 0.6398713 −0.12257113 −0.35854304
−2.10206795 −0.62725216 0.443176 0.23780456 −0.36947268 −0.39394468
−0.4086031 −0.51584613 1.40086424 0.15308252 0.08314608 −0.08646452
−0.08323111 0.34710139 −1.19955456 0.24803035 0.30780032 −0.18530244
0.37695128 1.03207827 0.40548515 −0.51752335 −0.06452624 1.02432513
−0.21949634 −0.54459727 0.25696743 −0.02352114 0.26655868 −0.10769007
−0.59957105 −0.00927553 0.14879346 0.27902412 −0.10014868 0.58578128
−0.44716626 −0.11416971 −0.3134813 −0.50771135 0.49232998 −0.87239206
−0.1993354 −0.64023274 0.00777942 0.53587562 0.35698465 0.35258824
1.74645114 −0.36962229 0.17113139 0.28024212 −0.04242261 −1.09624648
0.35567668 0.0048491 −1.41987002 −0.34854937 −0.37696782 −0.14530812]
[1.3104651 0.89598525 0.85248548 −1.4066149 −0.61097515 0.486164
0.04345426 −1.14857578 0.43672067 0.09375505 1.15657341 −0.20034783
−0.62035501 −0.06969572 −0.07186314 0.34525222 0.20739028 0.18144707
0.01666772 −0.46918312 0.98430043 −0.25594878 0.40150774 −0.18113485
−0.19719471 0.59473485 −0.22499575 0.56971848 0.7695933 0.04056893
0.53718734 0.99236411 0.52992195 0.628214 0.1834662 0.76305157
−0.24181075 0.89097691 0.55868387 0.12299256 −0.20449767 0.1152811
0.21897303 −0.4372482 −0.33119982 0.86824489 −0.40184823 0.94441861
−1.31633246 −0.26326489 0.11331466 −0.43175325 1.63447332 −0.50356722
−0.064597 −1.24537671 −0.05188338 −0.32861325 −0.00733493 0.53258693
1.38302946 0.22428341 0.57716918 0.11753339 −0.30517161 0.81589717
−0.43370757 −0.57834941 −1.00619471 −0.15559751 −0.48957777 −0.18705401]
[0.29579517 −0.39313304 0.22660404 −0.07769048 1.59768045 −0.75448525
0.11402565 0.42961523 1.05022395 −1.53991997 −0.00929202 0.1417277
1.45335686 −0.07969939 −0.77291811 0.32092518 −0.44920257 −0.84123546
−0.51920259 1.08528101 −1.47099924 −1.27706099 0.34755567 0.25915682
0.64393359 0.37473562 0.0528798 −0.62457448 −0.3748405 −0.47430333
−0.33036807 −0.7674076 0.12514977 −0.5085783 0.02627768 0.04784228
0.22703005 −0.91292828 −0.56555474 0.01274289 −0.79367 −0.27156559
0.04584835 1.05775762 −0.07219871 −0.02472326 −0.30923462 −0.98492271
0.3345578 0.20272861 0.05996652 −0.07382663 −0.72424048 0.61654133
0.40783596 0.20996921 0.52687436 0.52189082 −0.65698838 0.38132042
−0.34765172 0.04291533 −0.77955478 −0.10655218 1.59997082 0.25098574
−0.11212941 0.14723456 0.9638651 −0.45216152 0.36857039 0.47740829]
[−7.62265682e−01 −1.69236377e−01 −3.20280075e−01 −2.01835513e−01
1.31195617e+00 6.67759031e−02 −1.03672728e−01 4.55032110e−01
−1.85711771e−01 4.60101098e−01 5.76247334e−01 1.52587807e+00
1.60422134e+00 2.88353294e−01 −4.57065195e−01 −4.64541726e−02
−1.46385148e−01 3.58041257e−01 1.86820894e−01 1.85709685e−01
−5.26638746e−01 4.12468195e−01 −1.22571424e−01 −1.45727336e−01
−1.20605923e−01 −4.41346586e−01 3.90244722e−01 −5.31696796e−01
−4.85488474e−01 2.15438887e−01 −9.44682118e−03 6.81371847e−03
−2.69350290e−01 3.91651364e−03 8.35288107e−01 −3.92971396e−01
3.85517985e−01 −1.69645369e−01 −8.26047361e−03 −2.84272563e−02
−9.88544002e−02 1.24924920e−01 1.61330000e−01 −2.96193987e−01
1.88509598e−01 3.76898855e−01 3.14308465e−01 −4.16102529e−01
7.04013407e−01 −1.97082847e−01 1.61383048e−01 3.25785398e−01
−4.00427759e−01 5.35166681e−01 −7.74130523e−02 2.33510858e−03
2.36112654e−01 −2.21286356e−01 2.19614983e−01 −6.22923002e−02
−3.04360867e+00 −2.14334857e−02 −1.05726905e−03 −5.33363083e−03
−1.86623052e−01 1.07372916e+00 −2.98287272e−02 4.47440237e−01
5.26990116e−01 6.21019781e−01 8.67822673e−03 3.73851806e−01]
[1.01097715 −0.43187669 0.43921372 0.5321216 −0.34088054 −1.23371458
1.30667925 −0.46291575 −0.36887044 −0.42628133 −0.90258884 1.14848173
−0.67698836 −0.23642713 0.76691926 0.15012054 0.17513242 −0.23769177
−0.71736175 1.73428702 −0.72208363 −0.40311992 0.23535042 −0.40000367
−0.10122883 −0.26944473 −0.53836817 −0.9755494 −0.01715629 0.16589583
−0.35337713 −0.61074984 −0.13735074 −0.45587441 0.01250818 −0.39696515
0.26732519 −1.23419607 −0.38829738 −0.11969217 −0.58640665 −0.40061617
−0.24398375 1.00721014 0.73703206 −0.38457295 0.16650474 −1.99295473
−0.3466906 0.01132161 −0.93851066 0.90474617 0.36344501 −0.15687045
0.53075445 0.31692818 0.39067397 0.19387358 1.02101481 −0.19629753
−0.19112356 −0.14158687 0.09190591 −0.44029245 0.34570584 −0.14142781
0.17885733 1.1346246 −0.43545821 −0.04653888 −0.18016647 0.75078928]
[−0.40332806 0.15259214 −0.17188193 0.77254581 0.24573873 −0.16697757
0.03221319 −0.175248 −0.99172533 −0.48473996 −0.58634824 −0.002115
0.68528998 −0.00882054 −0.43505609 −0.11595152 −0.3524493 0.08346879
0.007276 −0.71797305 −0.67713475 −0.01415765 0.65797681 0.4020246
0.32403761 0.42483878 0.77840221 0.19374378 −0.97898698 0.23342216
0.46348882 −0.2604892 0.56031275 −0.38388926 0.51485229 −0.32569054
0.18112125 −0.05906491 −0.8849622 −0.54518116 0.01630897 0.07973013
0.10927051 −0.12975255 0.10494447 −0.16264115 0.08359384 0.46467236
0.79486549 −0.33018553 −0.51360339 0.19723307 −1.27263117 0.05408037
0.33270198 0.39826357 0.40751603 −0.73156345 −0.30242831 1.16275108
−0.71918142 −0.05969789 −0.55339873 0.50089788 1.19072211 0.57299292
−0.34327322 −0.12633982 −0.48915955 0.46941185 −0.43017337 −0.45001531]
[2.96421140e−01 −2.79853344e−01 −6.98803887e−02 5.05062819e−01
−5.51005125e−01 6.33165896e−01 1.43874735e−01 −1.08621635e−01
−3.32886875e−01 −4.16833729e−01 −9.43603516e−01 −1.16712379e+00
−3.87210041e−01 2.75952578e−01 −3.23279090e−02 −3.57172787e−01
4.36249644e−01 −1.82861071e−02 2.58781672e−01 −5.33354998e−01
1.28939116e+00 2.60455441e−02 −2.35185766e+00 −1.27888741e−02
−1.47248730e−01 1.31935686e−01 3.03985029e−01 −4.66788143e−01
−2.11565465e−01 −2.27709636e−01 −3.41227561e−01 2.38548517e−01
−2.74250418e−01 4.00132418e−01 −8.87395620e−01 2.53881305e−01
−1.53749129e−02 2.12793425e−01 −1.34601984e−02 3.53849798e−01
6.22939467e−02 −4.20864783e−02 1.22454733e−01 1.67652145e−01
2.35531822e−01 −4.46737036e−02 8.94509032e−02 9.69442190e−04
−8.11464012e−01 9.53373909e−02 −4.03275341e−01 3.73850465e−01
9.44294393e−01 2.36005902e−01 5.45237213e−02 9.00454968e−02
8.93701538e−02 4.87553120e−01 −3.14372689e−01 −1.52020361e−02
−2.07405433e−01 −5.78198671e−01 5.75446896e−02 1.07672743e−01
−1.29319906e+00 −8.85454416e−01 7.23549202e−02 6.12369478e−01
1.62590206e+00 1.58990562e−01 1.51147938e+00 −2.11744189e−01]
[9.27856445e−01 7.66313002e−02 3.99172276e−01 −4.20185536e−01
−1.50987637e+00 −6.46596551e−02 6.74637556e−01 −1.84285474e+00
4.42706615e−01 −1.06646553e−01 2.13650838e−01 −3.52122712e+00
−6.32900894e−01 −7.12150514e−01 1.49370044e−01 3.56142670e−02
9.39115644e−01 5.23202598e−01 −3.25768292e−01 1.60014676e−03
−2.49799445e−01 6.42951906e−01 −7.89430916e−01 −3.23023677e−01
4.11162227e−01 1.96627751e−02 −3.10375124e−01 −2.71319330e−01
6.79014385e−01 −2.49298140e−01 −1.14747250e+00 2.39820927e−01
−4.27306384e−01 −5.28884709e−01 −5.28806090e−01 −1.70187637e−01
8.03054348e−02 −2.92804867e−01 −4.42207098e−01 −2.86383301e−01
−4.30964202e−01 1.63471484e+00 −2.63395101e−01 2.06154168e−01
−4.38013002e−02 −1.15352303e−01 −1.78752422e−01 −7.15600491e−01
−9.81449366e−01 −1.10099459e+00 −3.08593847e−02 2.04869792e−01
1.11921883e+00 −7.43904933e−02 4.21198875e−01 −1.16597116e+00
−5.64654350e−01 −4.65134919e−01 −1.76635422e−02 −1.02395117e+00
1.34367585e+00 1.47077847e+00 −4.02934074e−01 1.35173306e−01
−8.45189840e−02 3.80579680e−01 1.87003106e−01 −1.00565588e+00
−7.54816905e−02 8.54150116e−01 8.48582506e−01 −3.69098455e−01]
[−1.01652241e+00 −9.62538719e−01 −9.00537908e−01 −5.61755776e−01
3.06524605e−01 −3.82472694e−01 −5.34508884e−01 −6.06634617e−01
−5.46674788e−01 −1.66454756e+00 −1.23285376e−01 −1.22519219e+00
−4.50771987e−01 3.80403370e−01 −1.40169120e+00 −7.88235784e−01
−1.10339272e+00 −1.23294628e+00 −1.75479978e−01 −2.26146793e+00
−6.53577387e−01 −8.32847714e−01 4.52658124e−02 2.95591116e−01
1.95145346e−02 2.28967935e−01 5.24699017e−02 9.60099697e−02
4.89438087e−01 −2.79727876e−01 −2.22504124e−01 −1.37113765e−01
2.13196203e−01 3.25297803e−01 2.48196483e−01 4.25459504e−01
−3.31054360e−01 2.06305861e+00 −1.25814706e−01 3.73249263e−01
2.36246869e−01 −7.54099861e−02 −1.35995567e−01 2.43467148e−02
8.01579803e−02 1.57950878e−01 2.43925810e−01 5.98892689e−01
1.55283487e+00 6.56906724e−01 −3.03255975e−01 5.81498444e−01
1.10783386e+00 −5.61047792e−01 7.42845535e−01 1.21913993e+00
5.67694664e−01 6.35293663e−01 −2.57383421e−04 −6.99199021e−01
−4.56939787e−01 −4.78504658e+00 −4.15146381e−01 −5.04416406e−01
5.72127879e−01 1.60570264e−01 5.54476902e−02 2.03169179e+00
−7.35928118e−01 −1.17394543e+00 9.68217969e−01 −2.59291679e−01]
[−0.42687833 −0.48533356 0.64311302 −0.00929711 −0.19943283 0.06997008
−0.04602098 0.02026976 −0.40539569 0.64021051 −0.67223287 0.98729295
−0.13881938 −0.63866407 0.08288065 −0.01751624 0.4302004 0.35916704
−0.20413868 1.16996169 −0.83412993 0.06903787 −0.82936823 −0.18880145
0.02469767 −0.59816158 −0.28310612 −0.47379851 −0.00732012 0.202236
−0.20265433 −0.66983831 −0.8194983 −0.4553943 0.23170933 −0.57556766
0.1105756 −0.2590903 −0.12170109 −0.04825823 −0.17867449 −0.00426845
−0.46629891 −0.10607629 −0.24761692 −0.30681381 0.29236212 −1.43815613
−0.29787946 0.03671697 −0.23525648 0.7019366 0.37830096 0.11734983
0.23228277 −1.03427982 0.7463578 0.02377096 0.57991886 0.08017296
−0.53541315 −0.57585174 −0.03689142 −0.32686636 0.34946737 1.03314221
1.12092638 0.05950843 −0.24445423 0.33641499 −0.39757463 0.74932653]

TABLE 7
Exemplary GRU Bias Weights

[0.05278961 0.8316648 0.18863174 0.15033871 0.27827257 0.41793922
0.57768679 −1.21303177 0.77303839 0.76739401 0.85747653 −0.96960849
−0.84362483 0.40098962 0.3029567 0.4747797 0.56314051 0.63889307
0.59328705 −1.14524996 0.31371805 −1.24521518 −0.19360186 0.40995541
0.20171499 0.39779955 −0.14729455 −0.25151581 0.71619838 −0.16598615
−0.21116211 0.19527936 0.32495099 0.14523123 −0.19277532 0.49395031
0.30553794 0.36637944 0.21195307 0.21741042 0.16961204 −0.27759224
−0.14555183 0.4773553 0.14465441 0.46810472 0.59798068 −0.21843031
0.20316558 0.4001261 0.20332371 0.48410556 −0.37445039 −0.15589076
0.60056067 −0.01115234 −0.09433793 0.49610326 0.28179428 −0.56945819
0.87747741 0.18429506 −0.69986153 −0.74835938 0.58253837 0.27031344
0.16094515 −0.47458473 −0.10866487 −0.49758652 0.08461624 0.16237779]

TABLE 8
Exemplary Dense Kernel Weights

[[−2.61987209], [−2.74449182], [1.11579788], [0.424615], [−1.54924107],
[−0.04262964], [−1.19196582], [1.87488842], [−1.04604852], [−9.520504],
[−0.42973623], [0.83459908], [−0.84549695], [0.98785937], [0.81342566],
[1.24084723], [−4.56023645], [2.00506091], [−0.33445692], [0.34981358],
[0.17092018], [3.64918661], [−1.02330852], [1.15306532], [−1.10116065],
[−4.60539246], [−2.0353477], [−0.26685116], [−0.43697673], [−0.02701966],
[−0.90783131], [1.13423789], [−0.11875227], [−1.20163226], [−0.264653],
[0.49540833], [−0.54647112], [1.39581072], [1.07455671], [1.8954289],
[−1.15799952], [1.83935165], [−0.25096184], [0.86369032], [3.87999415],
[5.12127209], [−0.82019556], [0.51716977], [−0.01084814], [−1.16863859],
[−2.65588641], [−0.56176919], [0.91245377], [1.88741946], [0.35669455],
[0.32712373], [0.96802062], [−3.62845445], [−0.98567683], [1.39873981],
[0.61592406], [−0.76576048], [1.30125141], [7.63295984], [−0.47387618],
[2.27207685], [0.04390277], [−0.01386968], [0.9903121], [1.17716086],
[0.92769176], [−0.75646734], [−0.82060498], [−3.3544867], [4.83388138],
[−0.98645198], [−0.03736724], [0.93488675], [0.49694148], [−1.1319176],
[−1.57193756], [−5.51461124], [−1.27979553], [−1.23176432], [1.65386283],
[−0.34214163], [2.10692334], [6.43121481], [0.28802058], [−0.18974693],
[−0.37807053], [−1.73556519], [−4.9620018], [1.28099215], [0.55229443],
[−0.23881915], [−1.45981669], [−3.53123856], [1.60507727], [−0.20383722],
[−0.83096808], [1.02352011], [−0.62400275], [−0.83913308], [−0.33934006],
[−0.72030431], [−0.31500763], [1.44448769], [−0.4940652], [−0.12226626],
[1.69216013], [4.87012196], [−0.63661289], [−0.78679216], [−0.07578238],
[−0.76843625], [1.89766276], [1.16742826], [0.09025002], [−0.56830335],
[−0.37783566], [−3.83753896], [1.0564152], [1.51446342], [1.20394123],
[−1.29641664], [0.03499955], [−0.03078461], [−1.61293674], [−4.13008451],
[−0.77203447], [0.02689628], [9.03535938], [−1.59541059], [1.56882], [1.48792505],
[−0.78430974], [1.53164887], [3.29070783], [0.08295513], [−4.28223944],
[0.754381], [1.16305184], [−2.35909033], [1.26050425], [−0.82405341],
[1.54408801], [−1.01838267], [3.32417297], [2.95703411], [−1.63332224],
[0.43653154], [−1.5520519], [−1.24083638], [−3.1849277], [−0.30942652],
[0.71937811], [−2.14475703], [0.69018376], [1.68623936], [−1.96524751],
[−0.60498351], [−1.98355019], [0.11083479], [2.46712756], [−1.12433839],
[3.64418125], [−2.39640927], [−0.51956719], [−0.24291636], [0.57978523],
[−6.0945673], [0.10263729], [−0.27764162], [−2.72303581], [−0.16383332],
[−1.99056792], [−1.22110534], [0.13481022], [0.07269326], [2.10644221],
[−0.20485984], [1.32259238], [0.4389846], [−0.53294241], [−2.99832201],
[−0.54540795], [−0.39156732], [−0.14623606], [−0.08744679], [0.11219146],
[−0.67484808], [0.08186885], [−0.4487884], [1.87775087], [−0.55267417],
[0.94654131], [−0.1756513], [−1.06539738], [0.31323799], [−3.86752582],
[0.17110589], [−1.45728362], [−0.45552397], [5.43897533], [−2.14521527],
[0.29699063], [−0.94607681], [−1.62727571], [−6.88942146], [−0.78646976],
[0.13265932], [3.10222387], [−0.4288899], [0.81925541], [−3.78744197],
[−3.26071548], [−3.68242168], [0.43415144], [−0.34977186], [−0.06043019],
[0.17738846], [−0.5465852], [−1.38373315], [−0.10127456], [0.13014296],
[−0.58907717], [4.05580521], [0.12054773], [0.17320366], [0.23538788],
[2.35936785], [−0.18914996], [0.08245806], [0.10508766], [−2.09618235],
[0.0818246], [0.94054776], [−0.04360234], [0.02606783], [0.53076208],
[−0.19547375], [1.89886475], [−0.36621055], [3.04003811], [2.37531924],
[−1.22876751], [0.79317045], [−0.58070278], [−0.57431567], [−2.58449435],
[−0.78229278], [0.54274672], [−2.57654619], [0.43814453], [1.204983],
[−1.41688418], [−0.75638103], [−2.43902469], [0.4760128], [1.95320261],
[−1.33007789], [3.02125597], [−2.77801085], [−0.52850872], [−0.79058319],
[3.81536555], [0.17168881], [0.26707801], [−0.11666939], [−0.14431302],
[−1.11402345], [−2.62366915], [−0.40183759], [−1.26764226], [7.66590643],
[0.49939862], [0.21904017], [0.3204582], [1.11153769], [−0.57232797],
[−2.1875608], [0.70980692], [−1.84378922], [2.8769362], [3.35384989],
[0.28388926], [−0.20034115], [0.45936164], [−1.99796987], [1.658692],
[−0.32202187], [1.63996577], [2.04414964], [−0.98592347], [0.16707386],
[−0.97844088], [−2.14522386], [−1.89455605], [1.11099887], [0.25790876],
[−1.21096063], [1.12859249], [2.05572748], [−1.2415905], [0.35611704],
[−1.6761024], [−1.19024849], [1.50422835], [1.62029195], [1.99725163],
[−1.36600935], [−0.10421184], [−0.63675714], [1.37432611], [−1.17094696],
[2.11921716], [1.76465583], [−1.50902295], [0.14934312], [−1.37046707],
[−1.03633738], [−1.69265819], [−0.10423797], [1.16956127], [−1.61897051],
[1.24858761], [1.18142843], [−1.39336586], [−1.95624554], [−1.44164801],
[−0.27539152], [1.5861702], [−0.55186683], [2.05847859], [−1.97251999], [−1.655357],
[−1.2858187], [0.58016235], [0.20358095], [1.12201607], [2.61793518],
[−0.16305976], [−0.63408267], [−0.18076663], [0.52133149], [−1.49981594],
[0.50581312], [−0.5879283], [−0.76506054], [−0.41027725], [0.90848994],
[−0.07201317], [−0.60734886], [−0.86664218], [−1.09011519], [3.49839711],
[0.02481496], [0.94401944], [−0.88661802], [0.79305601], [−0.05617219],
[−3.35063839], [−0.96625823], [1.2701633], [2.53422594], [−1.93773961],
[0.86408055], [−0.47361478], [−0.99658257], [0.63893008], [−0.25355098],
[−4.3421526], [−0.76761627], [0.86787891], [6.3512845], [−1.76245749],
[0.28173539], [−1.78608871], [0.71908641], [2.46769142], [−0.48900241],
[1.25488508], [−0.37793943], [−0.49508774], [−1.85472083], [1.55698836],
[0.12439785], [−0.17582354], [0.79157305], [−0.38698652], [−0.36067295],
[−1.85304523], [−0.69637132], [−0.60238278], [0.47999412], [4.53087473],
[−0.09689916], [0.15615597], [0.4993138], [0.1502312], [−0.89351928],
[−0.19223709], [−0.4449518], [1.46164191], [0.59800684], [−0.16509677],
[−0.11383091], [−1.90010691], [−3.62561083], [−0.33352557], [−0.78283554],
[−0.84435731], [−0.53739351], [−0.46635529], [−1.24566448], [0.13304973],
[−0.77858311], [0.30017087], [3.35144091], [0.37865117], [0.867351],
[0.55000478], [0.93095338], [−0.02101501], [−0.2429767], [0.38213503],
[−1.55181575], [−0.70577377], [1.15093338], [−0.95525289], [0.55708629],
[−2.71066093], [0.16160634], [−0.90091974], [−0.39295763], [0.92983431],
[0.33587086], [2.22746968], [−0.69766706], [3.4253056], [1.21584284],
[−0.43135348], [5.070889], [3.47405028], [−1.97454345], [1.04800975],
[1.84420216], [0.05070657], [−4.30840397], [−0.95053297], [−4.61424398],
[−2.13550138], [2.31353855], [0.84956491], [−3.3462019], [0.10757424],
[−2.41700411], [3.06325984], [0.05413959], [−0.2906431], [−0.16439679],
[−0.37667251], [−2.80349493], [1.07022965], [−0.45919123], [0.28715065],
[3.51798606], [−0.20659873], [0.4231495], [−0.04337826], [0.68107599],
[0.52428561], [−1.11207342], [0.54305077], [−5.80561781], [−1.07931507],
[5.10490465], [−0.36061084], [0.49795166], [−0.75417781], [−0.16746305],
[−0.05606421], [−5.40619612], [0.50346118], [1.27725101], [−3.77927661],
[−0.59508151], [−2.92244124], [−0.64737946], [−0.03244495], [0.36286822],
[2.59942985], [−1.06306911], [1.87645864], [1.00362885], [−1.26321745],
[−1.64785659], [−0.67570007], [−2.8217535], [−0.41199991], [0.35968539],
[−0.14602228], [−3.27993608], [−0.23458083], [−3.90086007], [0.8712939],
[−1.75339389], [0.49042204], [0.31142384], [−2.06346869], [0.1062976],
[−5.34781218], [−6.58793545], [−0.75038898], [−0.72449648], [−0.65361196],
[−1.12677944], [1.70866871], [2.2601378], [−2.59556556], [−2.54946876],
[−1.34336448], [−0.34460849], [0.85860181], [2.9661324], [0.32412654],
[−0.63424772], [−0.45634887], [−3.98189282], [0.51283479], [−2.345855],
[0.55048436], [1.99773324], [0.8870976], [−2.79413557], [−0.4427658],
[−4.10098457], [−0.84087974], [4.20394135], [2.79415107], [−0.17818461],
[1.79646218], [3.62023664], [2.53057051], [−0.37653288], [−0.6560716],
[−4.96990585], [−0.83017159], [2.72322345], [0.51787609], [−0.31166506],
[0.01790029], [−4.279706], [4.61982155], [−0.61431247], [0.65674198],
[1.79998755], [−1.66966128], [−0.16917685], [−1.52780712], [−2.02477217],
[−1.40718794], [2.13614631], [0.50742686], [−1.22540271], [0.32966271],
[3.80252433], [−1.07361615], [−0.44853446], [−1.18353879], [−0.31358987],
[7.80779839], [1.58769262], [0.39985418], [−0.80109018], [−0.51399338],
[−0.02393211], [−0.11496368], [−4.2544589], [0.37453282], [0.84448606],
[−3.28198862], [−0.15089157], [−6.33130074], [−0.3459048], [−0.54752886],
[1.55546105], [0.91082019], [−1.02209735], [1.75485933], [0.39727718],
[−1.96252489], [−2.61100173], [−0.30592236], [−0.78934699], [0.69529587],
[−3.19607663], [−0.05958025], [−3.77893949], [−2.05062556], [−0.07868424],
[1.41781664], [1.06674409], [−0.07583121], [−1.06082237], [−0.40827265],
[−1.94422483], [−2.38006234], [1.66149032], [−0.58137757], [1.76250386],
[1.70259798], [0.42165089], [−0.39305994], [−2.46295595], [−0.88196492],
[−5.64132166], [0.2809957], [−1.36836779], [6.84761572], [0.59932721],
[−0.65040511], [−0.7077145], [−0.0466348], [−6.32433796], [1.8601445], [−1.15626013],
[−0.86685377], [−0.11704612], [−2.10401893], [−1.10873604], [−2.49095106],
[−3.8402791], [−0.24776436], [0.39448604], [−0.39576483], [0.55474794],
[1.59122932], [1.16117358], [−3.45119977], [0.86734843], [0.23221132],
[−1.82093763], [1.92964971], [3.73869944], [−0.90630364], [1.75979996],
[0.08895387], [−1.20837176], [1.6286093], [−2.77600265], [1.4367491],
[1.68301201], [−4.49348974], [0.33018684], [−6.39774799], [−2.69219661],
[−1.07442737], [0.36035079], [−0.2515991], [−1.27690279], [2.05466318],
[1.14215207], [−3.98464346], [−2.56493998], [−1.2975378], [0.13554025],
[1.05732393], [0.56834555], [0.46816415], [−3.44895411], [0.09501681],
[−7.29191923], [0.81915492], [−0.73819566], [1.899526], [2.24298787],
[−0.12217813], [−1.58245409], [−0.15368246], [−6.88423157], [−1.85719323],
[2.390347], [−0.20369491], [0.85661167], [1.00321078], [4.36240482],
[−0.51789469], [4.23382854], [−0.64862293], [−1.48957205], [3.0601387],
[3.52824187], [0.37565956], [1.82035935], [−2.17702818], [−1.74509954],
[0.81387675], [−2.10778189], [−0.22873506], [0.61536282], [−1.98092914],
[−1.13677144], [−1.71172285], [−1.62906146], [−0.87260342], [1.25448585],
[1.38665819], [−0.01943529], [2.07292962], [1.53359985], [−1.91238344],
[−1.85476422], [−0.37645435], [−1.4104656], [1.71016824], [0.70332116],
[−0.53424418], [−0.62225986], [0.01264831], [−1.85801125], [2.5435226],
[−1.04375148], [1.37388206], [1.75628126], [−3.37509155], [0.21094686],
[−3.40618348], [−1.21254492], [−1.65779769], [−0.48687699], [−0.0758352],
[−1.23485005], [0.95099205], [0.01246844], [−3.48192239], [−1.46811819],
[−1.38792419], [−0.23289181], [2.86603475], [0.07756428], [1.36164784],
[−2.08329606], [−0.14057159], [0.50420427], [4.14942074], [−0.46483621],
[1.65599084], [1.65807486], [−0.73866171], [0.55337203], [−0.12334365],
[0.6970616], [−4.64469481], [−3.83106041], [1.2738266], [−2.6291399], [0.39295509],
[−2.15891314], [−0.60435939], [−0.71865606], [−2.17989397], [0.42331469],
[2.26066518], [−0.94228727], [2.83634591], [−5.82535744], [−0.4689683],
[−3.87215137], [−1.08274758], [−0.2872338], [−3.90575933], [3.56923175],
[0.63143402], [−1.1208818], [0.86022663], [−4.08034992], [0.21391267],
[6.37298918], [1.864838], [−2.82362866], [0.46120888], [4.15233231],
[1.40380359], [0.09902371], [−1.74923229], [−0.49445149], [−1.77451217],
[4.08006144], [0.01090435], [1.57321358], [−2.28478909], [−3.57266784],
[2.1245091], [−2.7657156], [−0.1984401], [−0.115915], [−2.8323648], [0.08341952],
[−2.5261445], [1.16862953], [0.30633542], [2.60291219], [1.80016315],
[−0.80374277], [0.10987681], [2.43843651], [−0.7505309], [−3.38618088],
[0.29461795], [−6.28850555], [1.7410295], [2.69255114], [−0.91341674],
[−1.30365932], [−0.85741848], [−1.47890019], [0.888722], [−5.83952141],
[−0.27224571], [0.20085666], [−1.26269841], [−0.11570544], [−2.20125866],
[−5.59145975], [−0.35150877], [−0.1843586], [5.11192274], [−0.02841036],
[4.24847698], [1.53836191], [−0.49722862], [−3.62489939], [−0.46835375],
[−0.20153835], [−1.19783223], [0.51044714], [−0.05640683], [−0.4295069],
[−0.51040554], [0.25671181], [1.68682373], [−1.23642421], [0.32745683],
[0.41564801], [−7.00627899], [0.12246624], [−8.92133999], [−0.11432713],
[−0.61936235], [0.75722575], [2.78831911], [0.56901509], [0.86331517],
[0.28973868], [−3.93950152], [−1.80874407], [−0.29313874], [0.41372204],
[1.13718736], [−0.43172848], [−0.22957496], [−1.18331814], [−0.09722157],
[−0.07029192], [0.80377942], [0.22125024], [4.62316704], [4.59250259],
[−0.32381529], [0.21349373], [−0.35275096], [−0.79100424], [−3.63003302],
[3.68920946], [−0.61196834], [−1.39465809], [0.24993621], [1.23726082],
[−0.34596285], [−0.13702109], [−9.73185539], [−0.37906736], [1.26818407],
[−0.88669145], [1.56380093], [−4.99425316], [−0.36970943], [−3.80218291],
[1.29774249], [−2.5686152], [0.76044399], [0.89938438], [−5.24779558],
[−0.17965518], [−5.89907646], [−3.55357718], [−0.63399416], [−0.60023171],
[−0.19020106], [−1.13860011], [1.980636], [0.95578218], [−1.27253687], [−4.0861578],
[−0.80701768], [−0.79534543], [0.83933783], [0.63074034], [0.23030068],
[−1.92293739], [0.1934866], [−1.69780946], [1.59376204], [−0.87825596],
[1.23192704], [2.63693404], [−2.88006616], [0.71512794], [−1.62760127],
[−1.49894392], [−2.21461773], [0.65598375], [−3.05315971], [−1.6988349],
[0.96423674], [2.6659205], [−2.88481951], [0.28735751], [−2.25478625],
[0.3760435], [5.72088957], [−0.17132981], [1.12482309], [−2.56077743],
[0.44371131], [0.36692384], [1.10967803], [0.12229718], [3.27048588],
[2.22869802], [−0.14119086], [1.01567328], [1.20322657], [−0.09340754],
[−2.00874043], [−1.3423202], [1.62868786], [−2.99753904], [−0.06213507],
[1.32903957], [0.23215015], [−0.12631455], [−2.37444639], [0.39509186],
[−0.17557953], [−2.25162292], [3.30790353], [−2.87937331], [0.07768845],
[−1.2067802], [1.12760532], [−0.50303227], [0.77229166], [2.81836367],
[−1.64538097], [0.36358249], [−0.58098668], [−1.01112056], [−1.48768437],
[0.54332238], [−4.71306801], [−0.86349106], [0.6293757], [2.6397984],
[−1.60149157], [1.14256191], [−2.51724911], [0.12252731], [5.71784639],
[0.09456697], [0.84209824], [−0.87289512], [0.00295472], [−0.51260436],
[−3.41061258], [−1.18450928], [3.08622527], [3.52076697], [−0.55116004],
[0.69805366], [5.62121582], [−1.62282813], [0.37544253], [0.258688],
[−5.44949102], [−1.65166056], [1.28007352], [5.13010311], [−0.04011408],
[5.03713989], [−2.74050999], [−0.27140597], [−4.34675503], [−0.8038708],
[4.48534584], [−2.26270819], [0.41852117], [−2.59040332], [3.49237871],
[0.12642422], [1.05143201], [3.77111244], [−1.51814103], [0.58842725],
[−2.87503052], [−1.35204422], [−1.38201749], [−0.32462415], [5.14712048],
[−0.67147142], [−0.05303688], [1.00022769], [−0.48595247], [−1.10384595],
[−0.89428312], [0.39385152], [6.35287809], [0.16479529], [1.12626326],
[0.65546256], [0.19661132], [0.27798921], [3.38115835], [0.11677053],
[1.11811996], [2.52084064], [−0.5575453], [0.39973536], [−0.1093071],
[−0.08769645], [−7.57875299], [−0.14282422], [−0.87788779], [−1.8052659], [0.048721],
[−0.6792751], [−0.27910811], [−0.16572276], [−5.27727699], [0.24314481],
[0.81775355], [−0.94828755], [1.16721117], [−3.98741841], [0.32078597],
[−0.84505957], [1.68198621], [−1.22913766], [0.88186198], [1.656214],
[−2.80722284], [0.4785611], [−3.61924839], [−0.29152998], [−1.01517057],
[−0.59552258], [0.62902552], [−1.14235866], [0.50269866], [−1.57375431],
[−3.19131351], [−3.68414068], [−2.11247444], [0.37717164], [5.8262887],
[−0.39856154], [0.94445789], [−1.68269193], [−0.4325594], [0.03961899],
[6.43392277], [−0.50738847], [1.32101643], [0.31589898], [−1.17276728],
[−0.0911948], [−0.77446729], [0.18687382], [−4.62173843], [−0.39241549],
[4.02061081], [−3.05401516], [0.47394723], [−3.80070662], [−0.80750769],
[−1.03881264], [−4.17408085], [−0.22195159], [2.15728951], [−0.46225539],
[0.50426018], [−5.36998367], [−0.00677548], [0.18166901], [1.66461027],
[0.30045786], [0.5772928], [0.02150481], [−1.21431923], [0.17602038],
[−3.57227421], [−0.46703953], [−1.63983655], [−0.14941879], [4.33673859],
[−0.96765935], [0.27484056], [0.79798764], [−4.60817957], [−6.52404737],
[−1.25777531], [0.01215173], [3.24317741], [−0.06969346], [0.57256693],
[−0.89470345], [−2.68595719], [−1.56258011], [0.3540332], [−2.14263964],
[−1.24158776], [0.1314192], [−1.69050658], [−1.75222158], [−0.99162811],
[−1.6769191], [−0.38416451], [2.24943566], [1.97233725], [0.33420476],
[2.32137299], [1.63776684], [−1.30133617], [−1.3751303], [−0.07562585],
[−2.31727982], [1.17262065], [1.05373263], [−1.25361073], [−0.01680006],
[−2.91758943], [−2.2703352], [0.55306691], [−1.98275661], [−1.16824424],
[0.08734406], [−1.53002036], [−1.47923505], [−0.98396158], [−1.66839933],
[−0.6729157], [2.17848182], [1.90584135], [0.22608781], [2.04538083],
[1.42320478], [−1.34839904], [−1.30217314], [−0.22820438], [−2.21097183],
[1.10826159], [1.16053128], [−1.0012331], [−0.05538922], [0.21386881],
[−3.63972068], [2.56678438], [−1.31732023], [1.95983267], [2.51462245],
[−3.33417892], [0.25402048], [−4.3701334], [−2.39910889], [−2.1261282],
[−0.13687438], [−0.82625818], [−0.7171194], [0.96590495], [1.40747225],
[−3.82771683], [−1.93615162], [−1.29935718], [−0.22092685], [3.61314845],
[0.74989849], [1.89349961], [−2.10083055], [−0.71272182], [−1.18278074],
[0.02569004], [−4.56284428], [−0.46944275], [0.72505933], [−4.32649183],
[−0.56127137], [−2.10300636], [−2.80426812], [0.05940181], [0.6700415],
[0.8503148], [0.18928875], [3.1236918], [2.39918566], [−2.24671626],
[−1.84000218], [−0.36967018], [−0.57524031], [−1.47373796], [0.65436858],
[−0.56387192], [−0.29511175], [−2.86063552], [−2.21933246], [0.09463561],
[−1.35220182], [−0.35154492], [0.00111112], [−1.25415576], [−1.47332346],
[0.34071746], [−1.22154212], [0.56134212], [3.4883244], [0.6929099],
[0.40520298], [1.01847529], [2.38931131], [−0.44533515], [−1.50836515],
[0.25682145], [−4.32589626], [−0.98919445], [2.56946039], [−0.55754423],
[0.2086709], [−1.23046219], [−5.48442888], [0.9006753], [−0.54097879],
[−0.69317597], [−0.23303831], [0.66852212], [−1.13535571], [0.49600986],
[−0.96282029], [0.90760577], [4.14376926], [1.90381491], [0.74935067],
[1.09037447], [1.75275326], [1.24110878], [0.65353626], [0.44779956],
[−9.36066914], [−0.67632788], [6.42790127], [−0.6395635], [1.15215635],
[−0.43144652], [−0.57796305], [0.14449762], [−4.41762686], [0.05648194],
[0.69972605], [−2.95838022], [−0.11526169], [−1.85737944], [−3.40667796],
[−0.33752206], [0.28825584], [1.40933812], [−0.15379809], [3.69857574],
[2.58826017], [−2.52796721], [−1.16477013], [−0.510903], [−0.33731398],
[−0.73834151], [0.34896955], [−0.04278188], [−0.4884488], [0.64670008],
[−3.04351068], [3.19855285], [0.18169364], [2.2983408], [2.54874444],
[−0.82667387], [0.86550826], [−0.3961601], [−3.41388154], [−4.85881758],
[1.2150712], [−1.02553153], [−2.59212637], [0.33813113], [0.67473108],
[−1.10222602], [0.3193751], [−3.81887412], [0.17340703], [3.82435918],
[1.26387227], [3.05783844], [−3.67877412], [−0.01971316], [−1.181162],
[2.84605432], [−1.24882114], [−2.00271559], [1.71017146], [−2.67017627],
[2.28081059], [−4.11373568], [−2.01315188], [−2.79283881], [2.88372135],
[1.33607197], [−3.25649452], [0.50101215], [2.05990505], [−2.4723165],
[−2.58668613], [−1.99001026], [−0.31186172], [1.00313759], [2.29015422],
[1.74497855], [−2.68916154]]

TABLE 9
Exemplary Dense Bias Weight

[0.37232602]

TABLE 10
Exemplary 4, 5 and 6 Hour MIC Determinations

| Dilution | 4 Hour Call | 5 Hour Call | 6 Hour Call | Target |
|---|---|---|---|---|
| 0 | 1 | 0.999984741 | 0.999999881 | 1 |
| 0.03 | 1 | 0.999991536 | 1 | 1 |
| 0.06 | 1 | 0.999980211 | 0.999999642 | 1 |
| 0.12 | 0.9999996 | 0.99999392 | 0.999998569 | 1 |
| 0.25 | 0.9999921 | 0.999997139 | 0.999995708 | 1 |
| 0.5 | 0.9999948 | 0.99999845 | 0.999998569 | 1 |
| 1 | 0.210478 | 0.939850688 | 0.951837778 | 1 |
| 2 | 0.0038356 | 4.89E−05 | 3.77E−06 | 0 |
| 4 | 0.0022583 | 7.26E−06 | 2.27E−07 | 0 |
| 8 | 0.0001531 | 0.000592581 | 0.000431988 | 0 |
| 16 | 0.0039348 | 0.001447048 | 2.32E−06 | 0 |
| 32 | 0.0096087 | 5.69E−05 | 1.23E−06 | 0 |
| 64 | 0.0011466 | 1.21E−05 | 1.64E−06 | 0 |

TABLE 11
Exemplary Antimicrobials and Antimicrobial Classes

| Antimicrobial class | Specific antimicrobial |
|---|---|
| Aminoglycoside | Gentamicin |
| Carbapenems | Meropenem |
| Cephem | Cefepime |
| | Cefotaxime |
| | Ceftazidime |
| Flouoroquinolones | Levofloxacin |
| Tetracyclines | Tetracycline |

TABLE 12
Exemplary Microorganisms and Microorganism Classes

| Microorganism Class | Specific Microorganism |
|---|---|
| Gram-Negative Fermenters | Citrobacter freundii |
| | Enterobacter aerogenes |
| | Enterobacter cloacae |
| | Eschericia coli |
| | Klebsiella oxytoca |
| | Klebsiella pneumoniae |
| | Proteus mirabilis |
| | Providendia rettgeri |
| | Salmonella paratyphi |
| | Serratia marcescens |
| Gram-Negative Non Fermenters | Acinetobacter baumannii |
| | Pseudomonas aeruginosa |
| | Pseudomonas fluorescens |

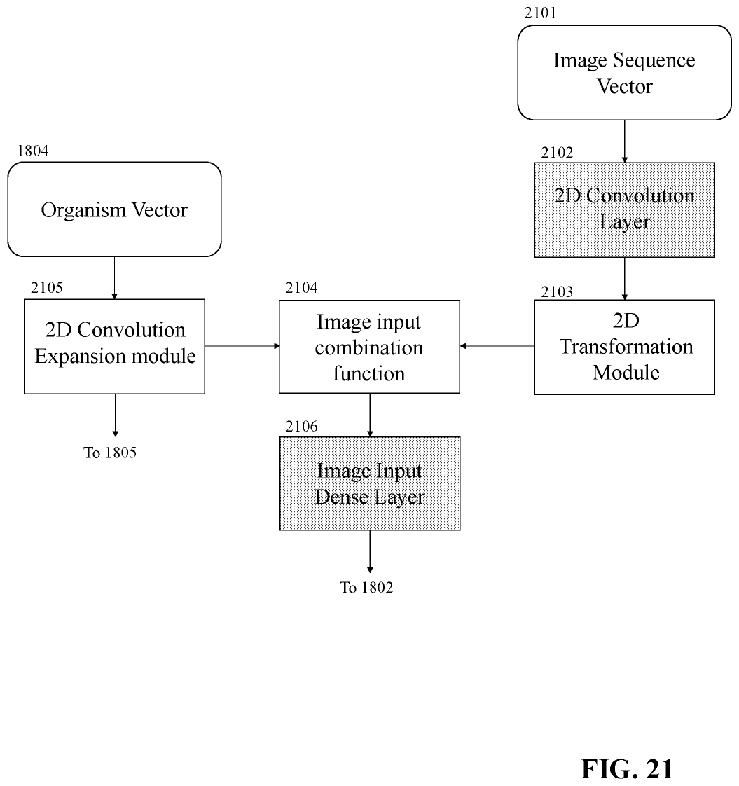

It should be understood that, while the above description has focused on use of machine learning models to make MIC determinations, some embodiments may use such models for other purposes as well. For example, in some embodiments, a model using the architecture of FIG. 18 and trained to make MIC determinations as described above could be used to generate new models for deriving microbial counts from images, or even to bootstrap a complete MIC determination system. To consider how this may take place, consider the architecture of FIG. 21, which shows how an architecture as illustrated in FIG. 18 could be modified to train a model to provide counts that could subsequently be used in MIC determination.

In the architecture of FIG. 21, rather than taking a growth sequence vector 1801 as input, input regarding a test well would be provided in the form of an image sequence vector 2101—e.g., a series of images of a test well such as could be provided by periodically imaging the test well as described herein. This image vector could then be processed by a machine learning model (e.g., a Keras API 2D convolution layer 2102) to extract salient features (e.g., signal strengths for how strongly particular pixel windows, such as 3×3 pixel windows in a N×N pixel image, match a particular pattern) from the images. This feature information could then be provided to a 2D transformation module 2103 which could put it in a form which could be combined with organism information in a manner similar to what was described previously in the context of FIG. 18 (e.g., by taking a global average of strength values from a Keras API 2D convolution layer 2102 to obtain a density value for an image, then duplicating that image to create a vector that could be combined with an organism vector as described previously). Meanwhile, the organism vector 1804, would be transformed with a 2D Convolution expansion module 2105, that could, in a manner similar to the expansion module 1805 of FIG. 18, duplicate the organism vector so that it is compatible for combination with the information from the image sequence vector 2101. The outputs of the convolution expansion module 2105 and the 2D transformation module 2013 could then be combined using an image input combination function 2014 (e.g., multiplication, concatenation), and the combined information could be fed to a dense layer 2106 adapted to provide a sequence of values that would take the place of the growth sequence vector 1801 in a model following the architecture of FIG. 18.

Using an architecture such as shown in FIG. 21, a model could be trained to generate microorganism counts by connecting the outputs to a model which had already been trained to make MIC determinations based on organism count information. The trainable portions of the MIC determination model could then be locked, and the count generation model could then be trained based on whether its output allowed the MIC determination model to make correct growth/no-growth determinations. Once the count generation model had been trained sufficiently to allow the MIC determination model to give reasonable answers, the trainable portions of the MIC determination model could be unlocked, and the combined model (i.e., count generation+MIC determination) model could be given end to end training as a further fine tuning measure.

### IV. EXEMPLARY COMBINATIONS

It will be appreciated that embodiments of the invention may be implemented using a variety of different information processing systems. In particular, although the figures and the discussion thereof provide an exemplary computing system and methods, these are presented merely to provide a useful reference in discussing various aspects of the invention. Embodiments of the invention may be carried out on any suitable data processing device, such as a personal computer, laptop, personal digital assistant, mobile telephone, server computer, etc. Of course, the description of the systems and methods has been simplified for purposes of discussion, and they are just one of many different types of system and method that may be used for embodiments of the invention. It will be appreciated that the boundaries between logic blocks are merely illustrative and that alternative embodiments may merge logic blocks or elements, or may impose an alternate decomposition of functionality upon various logic blocks or elements.

It will be appreciated that the above-mentioned functionality may be implemented as one or more corresponding modules as hardware and/or software. For example, the above-mentioned functionality may be implemented as one or more software components for execution by a processor of the system. Alternatively, the above-mentioned functionality may be implemented as hardware, such as on one or more field-programmable-gate-arrays (FPGAs), and/or one or more application-specific-integrated-circuits (ASICs), and/or one or more digital-signal-processors (DSPs), and/or one or more graphical processing units (GPUs), and/or other hardware arrangements. Method steps implemented in flowcharts contained herein, or as described above, may each be implemented by corresponding respective modules; multiple method steps implemented in flowcharts contained herein, or as described above, may be implemented together by a single module.

It will be appreciated that, insofar as embodiments of the invention are implemented by a computer program, then one or more storage media and/or one or more transmission media storing or carrying the computer program form aspects of the invention. The computer program may have one or more program instructions, or program code, which, when executed by one or more processors (or one or more computers), carries out an embodiment of the invention. The term “program” as used herein, may be a sequence of instructions designed for execution on a computer system, and may include a subroutine, a function, a procedure, a module, an object method, an object implementation, an executable application, an applet, a servlet, source code, object code, byte code, a shared library, a dynamic linked library, and/or other sequences of instructions designed for execution on a computer system. The storage medium may be a magnetic disc (such as a hard drive or a floppy disc), an optical disc (such as a CD-ROM, a DVD-ROM or a BluRay disc), or a memory (such as a ROM, a RAM, EEPROM, EPROM, Flash memory or a portable/removable memory device), etc. The transmission medium may be a communications signal, a data broadcast, a communications link between two or more computers, etc.

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method comprising: (a) creating a plurality of test mixtures in a plurality of test wells, wherein: (i) each test mixture from the plurality of test mixtures is inoculated using a biological sample; (ii) each test mixture from the plurality of test mixtures comprises an antimicrobial solution comprising an antimicrobial agent; (iii) in each test mixture from the plurality of test mixtures, the antimicrobial solution in that test mixture differs from the antimicrobial solution in each other test mixture from the plurality of test mixtures; and (iv) the same biological sample is used to inoculate each test mixture from the plurality of test mixtures; (b) incubating each of the test mixtures; (c) for each test mixture from the plurality of test mixtures, at a plurality of imaging times, wherein each of the imaging times takes place after incubation has begun, capturing an image of that test mixture; (d) obtaining a plurality of growth predictions by performing steps comprising, for each test mixture from the plurality of test mixtures, providing a data sequence to a machine learning model, wherein: (i) the data sequence comprises a plurality of input items; (ii) each input item from the plurality of input items corresponds to an imaging time from the plurality of imaging times; and (iii) the machine learning model is adapted to recognize, and to make growth predictions based on, temporal sequences; and (e) generating a MIC determination for the biological sample based on the plurality of growth predictions.

### Example 2

The method of example 1, wherein: (a) the machine learning model comprises: (i) a network cluster comprising a plurality of recurrent neural networks; and (ii) a dense layer comprising a feed forward neural network; (b) obtaining the plurality of growth predictions comprises, for each test mixture from the plurality of test mixtures: (i) providing a temporal sequence based on the data sequence for that test mixture to each recurrent neural network from the plurality of recurrent neural networks; (ii) obtaining, from each recurrent neural network from the plurality of recurrent neural networks, an intermediate growth prediction for that test mixture; and (iii) obtaining, from the dense layer, a growth prediction for that test mixture based on the intermediate growth predictions for that test mixture obtained from the recurrent neural networks.

### Example 3

The method of example 2, wherein: (a) the machine learning model is adapted to receive an identification of a microorganism corresponding to the biological sample; and (b) the dense layer is adapted to, for each test mixture, weight the intermediate growth predictions for that test mixture from the plurality of recurrent neural networks based on the identification of the microorganism.

### Example 4

The method of example 2, wherein the machine learning model is adapted to, for each test mixture from the plurality of test mixtures, generate the temporal sequence based on the data sequence for that test mixture by performing steps comprising: (a) for each imaging time from the plurality of imaging times, obtaining a doubling value by applying a log base 2 transformation to the input item corresponding to that imaging time comprised by the data sequence for that test mixture; and (b) for each doubling value except the doubling value corresponding to a first imaging time, obtaining a doubling value change by subtracting a doubling value corresponding to a preceding imaging time.

### Example 5

The method of example 2, wherein the plurality of recurrent neural network comprises 16 recurrent neural networks.

### Example 6

The method of example 5, wherein the plurality of recurrent neural networks comprises 24 gated recurrent units.

### Example 7

The method of example 1, wherein: (a) in each test mixture from the plurality of test mixtures, the antimicrobial solution in that text mixture has a corresponding antimicrobial concentration which is different from the antimicrobial concentrations which correspond to the other test mixtures from the plurality of test mixtures; (b) the plurality of growth predictions comprises, for each test mixture from the plurality of test mixtures, a growth prediction corresponding to that test mixture; and (c) generating the MIC determination for the biological sample based on the plurality of growth predictions comprises determining that a lowest concentration corresponding to a test mixture with a corresponding growth prediction of no growth as the MIC determination.

### Example 8

The method of example 7, wherein the plurality of test mixtures comprises a growth mixture, in which the corresponding antimicrobial concentration is no antimicrobial.

### Example 9

A biological testing system comprising a processor configured with a set of computer instructions operable, when executed, to cause the system to perform a method comprising: (a) creating a plurality of test mixtures in a plurality of test wells, wherein: (i) each test mixture from the plurality of test mixtures is inoculated using a biological sample; (ii) each test mixture from the plurality of test mixtures comprises an antimicrobial solution comprising an antimicrobial agent; (iii) in each test mixture from the plurality of test mixtures, the antimicrobial solution in that test mixture differs from the antimicrobial solution in each other test mixture from the plurality of test mixtures; and (iv) the same biological sample is used to inoculate each test mixture from the plurality of test mixtures; (b) incubating each of the test mixtures; (c) for each test mixture from the plurality of test mixtures, at a plurality of imaging times, wherein each of the imaging times takes place after incubation has begun, capturing an image of that test mixture; (d) obtaining a plurality of growth predictions by performing steps comprising, for each test mixture from the plurality of test mixtures, providing a data sequence to a machine learning model, wherein: (i) the data sequence comprises a plurality of input items; (ii) each input item from the plurality of input items corresponds to an imaging time from the plurality of imaging times; and (iii) the machine learning model is adapted to recognize, and to make growth predictions based on, temporal sequences; and (e) generating a MIC determination for the biological sample based on the plurality of growth predictions.

### Example 10

The system of example 9, wherein: (a) the machine learning model comprises: (i) a network cluster comprising a plurality of recurrent neural networks; and (ii) a dense layer comprising a feed forward neural network; (b) obtaining the plurality of growth predictions comprises, for each test mixture from the plurality of test mixtures: (i) providing a temporal sequence based on the data sequence for that test mixture to each recurrent neural network from the plurality of recurrent neural networks; (ii) obtaining, from each recurrent neural network from the plurality of recurrent neural networks, an intermediate growth prediction for that test mixture; and (iii) obtaining, from the dense layer, a growth prediction for that test mixture based on the intermediate growth predictions for that test mixture obtained from the recurrent neural networks.

### Example 11

The system of example 10, wherein: (a) the machine learning model is adapted to receive an identification of a microorganism corresponding to the biological sample; and (b) the dense layer is adapted to, for each test mixture, weight the intermediate growth predictions for that test mixture from the plurality of recurrent neural networks based on the identification of the microorganism.

### Example 12

The system of example 10, wherein the machine learning model is adapted to, for each test mixture from the plurality of test mixtures, generate the temporal sequence based on the data sequence for that test mixture by performing steps comprising: (a) for each imaging time from the plurality of imaging times, obtaining a doubling value by applying a log base 2 transformation to the input item corresponding to that imaging time comprised by the data sequence for that test mixture; and (b) for each doubling value except the doubling value corresponding to a first imaging time, obtaining a doubling value change by subtracting a doubling value corresponding to a preceding imaging time.

### Example 13

The system of example 10, wherein the plurality of recurrent neural network comprises 16 recurrent neural networks.

### Example 14

The system of example 13, wherein the plurality of recurrent neural networks comprises 24 gated recurrent units.

### Example 15

A computer program product comprising a non-transitory computer readable medium having stored thereon a set of computer instructions operable, when executed, to cause a biological testing system to perform a method comprising: (a) creating a plurality of test mixtures in a plurality of test wells, wherein: (i) each test mixture from the plurality of test mixtures is inoculated using a biological sample; (ii) each test mixture from the plurality of test mixtures comprises an antimicrobial solution comprising an antimicrobial agent; (iii) in each test mixture from the plurality of test mixtures, the antimicrobial solution in that test mixture differs from the antimicrobial solution in each other test mixture from the plurality of test mixtures; and (iv) the same biological sample is used to inoculate each test mixture from the plurality of test mixtures; (b) incubating each of the test mixtures; (c) for each test mixture from the plurality of test mixtures, at a plurality of imaging times, wherein each of the imaging times takes place after incubation has begun, capturing an image of that test mixture; (d) obtaining a plurality of growth predictions by performing steps comprising, for each test mixture from the plurality of test mixtures, providing a data sequence to a machine learning model, wherein: (i) the data sequence comprises a plurality of input items; (ii) each input item from the plurality of input items corresponds to an imaging time from the plurality of imaging times; and (iii) the machine learning model is adapted to recognize, and to make growth predictions based on, temporal sequences; and (e) generating a MIC determination for the biological sample based on the plurality of growth predictions.

### Example 16

The computer program product of example 15, wherein: (a) the machine learning model comprises: (i) a network cluster comprising a plurality of recurrent neural networks; and (ii) a dense layer comprising a feed forward neural network; (b) obtaining the plurality of growth predictions comprises, for each test mixture from the plurality of test mixtures: (i) providing a temporal sequence based on the data sequence for that test mixture to each recurrent neural network from the plurality of recurrent neural networks; (ii) obtaining, from each recurrent neural network from the plurality of recurrent neural networks, an intermediate growth prediction for that test mixture; and (iii) obtaining, from the dense layer, a growth prediction for that test mixture based on the intermediate growth predictions for that test mixture obtained from the recurrent neural networks.

### Example 17

The computer program product of example 16, wherein: (a) the machine learning model is adapted to receive an identification of a microorganism corresponding to the biological sample; and (b) the dense layer is adapted to, for each test mixture, weight the intermediate growth predictions for that test mixture from the plurality of recurrent neural networks based on the identification of the microorganism.

### Example 18

The computer program product of example 16, wherein the machine learning model is adapted to, for each test mixture from the plurality of test mixtures, generate the temporal sequence based on the data sequence for that test mixture by performing steps comprising: (a) for each imaging time from the plurality of imaging times, obtaining a doubling value by applying a log base 2 transformation to the input item corresponding to that imaging time comprised by the data sequence for that test mixture; and (b) for each doubling value except the doubling value corresponding to a first imaging time, obtaining a doubling value change by subtracting a doubling value corresponding to a preceding imaging time.

### Example 19

The computer program product of example 16, wherein the plurality of recurrent neural network comprises 16 recurrent neural networks.

### Example 20

The computer program product of example 19, wherein the plurality of recurrent neural networks comprises 24 gated recurrent units.

### V. MISCELLANEOUS

It should be understood that, in the above examples and the claims, a statement that something is “based on” something else should be understood to mean that it is determined at least in part by the thing that it is indicated as being based on. To indicate that something must be completely determined based on something else, it is described as being “based EXCLUSIVELY on” whatever it must be completely determined by.

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A method comprising:
(a) creating a plurality of test mixtures in a plurality of test wells, wherein:
(i) each test mixture from the plurality of test mixtures is inoculated using a biological sample;
(ii) each test mixture from the plurality of test mixtures comprises an antimicrobial solution comprising an antimicrobial agent;
(iii) in each test mixture from the plurality of test mixtures, the antimicrobial solution in that test mixture differs from the antimicrobial solution in each other test mixture from the plurality of test mixtures; and
(iv) the same biological sample is used to inoculate each test mixture from the plurality of test mixtures;
(b) incubating each of the test mixtures;
(c) for each test mixture from the plurality of test mixtures, at a plurality of imaging times, wherein each of the imaging times takes place after incubation has begun, capturing an image of that test mixture;
(d) obtaining a plurality of growth predictions by performing steps comprising, for each test mixture from the plurality of test mixtures, providing a data sequence to a machine learning model, wherein:
(i) the data sequence comprises a plurality of input items;
(ii) each input item from the plurality of input items corresponds to an imaging time from the plurality of imaging times; and
(iii) the machine learning model is adapted to recognize, and to make growth predictions based on, temporal sequences;
and
(e) generating a minimum inhibitory concentration, MIC, determination for the biological sample based on the plurality of growth predictions.

2. The method of claim 1, wherein:
(a) the machine learning model comprises:
(i) a network cluster comprising a plurality of recurrent neural networks; and
(ii) a dense layer comprising a feed forward neural network;
(b) obtaining the plurality of growth predictions comprises, for each test mixture from the plurality of test mixtures:
(i) providing a temporal sequence based on the data sequence for that test mixture to each recurrent neural network from the plurality of recurrent neural networks;
(ii) obtaining, from each recurrent neural network from the plurality of recurrent neural networks, an intermediate growth prediction for that test mixture; and
(iii) obtaining, from the dense layer, a growth prediction for that test mixture based on the intermediate growth predictions for that test mixture obtained from the recurrent neural networks.

3. The method of claim 2, wherein:
(a) the machine learning model is adapted to receive an identification of a microorganism corresponding to the biological sample; and
(b) the dense layer is adapted to, for each test mixture, weight the intermediate growth predictions for that test mixture from the plurality of recurrent neural networks based on the identification of the microorganism.

4. The method of claim 2, wherein the machine learning model is adapted to, for each test mixture from the plurality of test mixtures, generate the temporal sequence based on the data sequence for that test mixture by performing steps comprising:
(a) for each imaging time from the plurality of imaging times, obtaining a doubling value by applying a log base 2 transformation to the input item corresponding to that imaging time comprised by the data sequence for that test mixture; and
(b) for each doubling value except the doubling value corresponding to a first imaging time, obtaining a doubling value change by subtracting a doubling value corresponding to a preceding imaging time.

5. The method of claim 2, wherein the plurality of recurrent neural network comprises 16 recurrent neural networks.

6. The method of claim 5, wherein the plurality of recurrent neural networks comprises 24 gated recurrent units.

7. The method of claim 1, wherein:
(a) in each test mixture from the plurality of test mixtures, the antimicrobial solution in that text mixture has a corresponding antimicrobial concentration which is different from the antimicrobial concentrations which correspond to the other test mixtures from the plurality of test mixtures;
(b) the plurality of growth predictions comprises, for each test mixture from the plurality of test mixtures, a growth prediction corresponding to that test mixture; and
(c) generating the MIC determination for the biological sample based on the plurality of growth predictions comprises determining that a lowest concentration corresponding to a test mixture with a corresponding growth prediction of no growth as the MIC determination.

8. The method of claim 7, wherein the plurality of test mixtures comprises a growth mixture, in which the corresponding antimicrobial concentration is no antimicrobial.

9. A biological testing system comprising a processor configured with a set of computer instructions operable, when executed, to cause the system to perform a method comprising:
(a) creating a plurality of test mixtures in a plurality of test wells, wherein:
(i) each test mixture from the plurality of test mixtures is inoculated using a biological sample;
(ii) each test mixture from the plurality of test mixtures comprises an antimicrobial solution comprising an antimicrobial agent;
(iii) in each test mixture from the plurality of test mixtures, the antimicrobial solution in that test mixture differs from the antimicrobial solution in each other test mixture from the plurality of test mixtures; and
(iv) the same biological sample is used to inoculate each test mixture from the plurality of test mixtures;
(b) incubating each of the test mixtures;
(c) for each test mixture from the plurality of test mixtures, at a plurality of imaging times, wherein each of the imaging times takes place after incubation has begun, capturing an image of that test mixture;
(d) obtaining a plurality of growth predictions by performing steps comprising, for each test mixture from the plurality of test mixtures, providing a data sequence to a machine learning model, wherein:
(i) the data sequence comprises a plurality of input items;
(ii) each input item from the plurality of input items corresponds to an imaging time from the plurality of imaging times; and
(iii) the machine learning model is adapted to recognize, and to make growth predictions based on, temporal sequences;
and
(e) generating a minimum inhibitory concentration, MIC, determination for the biological sample based on the plurality of growth predictions.

10. The system of claim 9, wherein:
(a) the machine learning model comprises:
(i) a network cluster comprising a plurality of recurrent neural networks; and
(ii) a dense layer comprising a feed forward neural network;
(b) obtaining the plurality of growth predictions comprises, for each test mixture from the plurality of test mixtures:
(i) providing a temporal sequence based on the data sequence for that test mixture to each recurrent neural network from the plurality of recurrent neural networks;
(ii) obtaining, from each recurrent neural network from the plurality of recurrent neural networks, an intermediate growth prediction for that test mixture; and
(iii) obtaining, from the dense layer, a growth prediction for that test mixture based on the intermediate growth predictions for that test mixture obtained from the recurrent neural networks.

11. The system of claim 10, wherein:
(a) the machine learning model is adapted to receive an identification of a microorganism corresponding to the biological sample; and
(b) the dense layer is adapted to, for each test mixture, weight the intermediate growth predictions for that test mixture from the plurality of recurrent neural networks based on the identification of the microorganism.

12. The system of claim 10, wherein the machine learning model is adapted to, for each test mixture from the plurality of test mixtures, generate the temporal sequence based on the data sequence for that test mixture by performing steps comprising:
(a) for each imaging time from the plurality of imaging times, obtaining a doubling value by applying a log base 2 transformation to the input item corresponding to that imaging time comprised by the data sequence for that test mixture; and
(b) for each doubling value except the doubling value corresponding to a first imaging time, obtaining a doubling value change by subtracting a doubling value corresponding to a preceding imaging time.

13. The system of claim 10, wherein the plurality of recurrent neural network comprises 16 recurrent neural networks.

14. The system of claim 13, wherein the plurality of recurrent neural networks comprises 24 gated recurrent units.

15. A computer program product comprising a non-transitory computer readable medium having stored thereon a set of computer instructions operable, when executed, to cause a biological testing system to perform a method comprising:
(a) creating a plurality of test mixtures in a plurality of test wells, wherein:
(i) each test mixture from the plurality of test mixtures is inoculated using a biological sample;
(ii) each test mixture from the plurality of test mixtures comprises an antimicrobial solution comprising an antimicrobial agent;
(iii) in each test mixture from the plurality of test mixtures, the antimicrobial solution in that test mixture differs from the antimicrobial solution in each other test mixture from the plurality of test mixtures; and
(iv) the same biological sample is used to inoculate each test mixture from the plurality of test mixtures;
(b) incubating each of the test mixtures;
(c) for each test mixture from the plurality of test mixtures, at a plurality of imaging times, wherein each of the imaging times takes place after incubation has begun, capturing an image of that test mixture;
(d) obtaining a plurality of growth predictions by performing steps comprising, for each test mixture from the plurality of test mixtures, providing a data sequence to a machine learning model, wherein:
(i) the data sequence comprises a plurality of input items;
(ii) each input item from the plurality of input items corresponds to an imaging time from the plurality of imaging times; and
(iii) the machine learning model is adapted to recognize, and to make growth predictions based on, temporal sequences;
and
(e) generating a minimum inhibitory concentration, MIC, determination for the biological sample based on the plurality of growth predictions.

16. The computer program product of claim 15, wherein:
(a) the machine learning model comprises:
(i) a network cluster comprising a plurality of recurrent neural networks; and
(ii) a dense layer comprising a feed forward neural network;
(b) obtaining the plurality of growth predictions comprises, for each test mixture from the plurality of test mixtures:
(i) providing a temporal sequence based on the data sequence for that test mixture to each recurrent neural network from the plurality of recurrent neural networks;
(ii) obtaining, from each recurrent neural network from the plurality of recurrent neural networks, an intermediate growth prediction for that test mixture; and
(iii) obtaining, from the dense layer, a growth prediction for that test mixture based on the intermediate growth predictions for that test mixture obtained from the recurrent neural networks.

17. The computer program product of claim 16, wherein:
(a) the machine learning model is adapted to receive an identification of a microorganism corresponding to the biological sample; and
(b) the dense layer is adapted to, for each test mixture, weight the intermediate growth predictions for that test mixture from the plurality of recurrent neural networks based on the identification of the microorganism.

18. The computer program product of claim 16, wherein the machine learning model is adapted to, for each test mixture from the plurality of test mixtures, generate the temporal sequence based on the data sequence for that test mixture by performing steps comprising:
(a) for each imaging time from the plurality of imaging times, obtaining a doubling value by applying a log base 2 transformation to the input item corresponding to that imaging time comprised by the data sequence for that test mixture; and
(b) for each doubling value except the doubling value corresponding to a first imaging time, obtaining a doubling value change by subtracting a doubling value corresponding to a preceding imaging time.

19. The computer program product of claim 16, wherein the plurality of recurrent neural network comprises 16 recurrent neural networks.

20. The computer program product of claim 19, wherein the plurality of recurrent neural networks comprises 24 gated recurrent units.
